(19) United States
(12) Patent Application Publication
(10) Pub. No.: US 2005/0202515 A1
(43) Pub. Date: Sep. 15, 2005

(54) Functional method for generating or screening for ligands which modulate steroid hormone receptors

(75) Inventors: Frank Barletta, Hawthorne, NJ (US); Greg O'Donnell, Philadelphia, PA (US); Boris J. Cheskis, North Wales, PA (US)

Correspondence Address: DARBY & DARBY P.C., P.O. BOX 5257, NEW YORK, NY, 10150-5257, US

(73) Assignee: Wyeth, Madison, NJ (US)

(21) Appl. No.: 11/067,321

(22) Filed: Feb. 25, 2005

Related U.S. Application Data
(60) Provisional application No. 60/548,352, filed on Feb. 27, 2004.

(51) Int. Cl. G01N 33/53; G01N 33/567

(52) U.S. Cl. 435/7.2

(57) ABSTRACT

This application presents the discovery that the Vitamin D3 receptor (VDR), a member of the nuclear hormone ligand-activated receptor superfamily, interacts with a MNAR, a scaffolding protein. This interaction results in the formation of a ternary complex between VDR, MNAR, and the Src or PI3 kinase families of tyrosine kinases to mediate cell signaling, especially in osteoblast cells.

### FIELD OF THE INVENTION

The present invention provides a method of functionally designing, or identifying by screening, selective ligands of the Vitamin D receptor (VDR) which can be used for the treatment of osteoporosis and other disorders for which signaling through the VDR has been implicated. The invention relates to the discovery that VDR interacts with MNAR, a scaffolding protein previously demonstrated to interact with other nuclear hormone receptors, and that ligand-dependent interaction between VDR and MNAR leads to activation of Src/MAP kinase pathway.

### BACKGROUND OF THE INVENTION

Nuclear Hormone Receptors

Nuclear hormone receptors are a superfamily of ligand-inducible transcription factors, which, as a class, are involved in ligand-dependent transcriptional control of gene expression. Binding of a specific ligand, inducing conformational changes in the receptor molecule, affects receptor interaction with other transcription factors, and, ultimately, formation of the preinitiation complex. This process regulates the rate of gene transcription. (D. J. Mangelsdorf, et al., Cell 1995; 83: 835-9).

The nuclear hormone receptor superfamily includes steroid hormone receptors, non-steroid hormone receptors, and orphan receptors. The receptors for glucocorticoids (GR), mineralcorticoids (MR), progestins (PR), androgens (AR), and estrogens (ER) are examples of classical steroid receptors. In addition to steroid hormone receptors, this nuclear hormone receptor superfamily consists of receptors for non-steroid hormones, such as vitamin D3, thyroid hormones, and retinoids. Moreover, a range of nuclear receptor-like sequences have been identified which encode so called “orphan” receptors. These orphan receptors are structurally related to and, therefore, classified as nuclear hormone receptors, although no putative ligands have been identified yet. (Cell 1995, 83: 851-857).

The superfamily of nuclear hormone receptors is characterized structurally and functionally by a modular structure in comprising six distinct structural and functional domains, A to F. More specifically these receptors have a variable N-terminal region (domain A/B); followed by a centrally located, highly conserved DNA-binding domain (hereinafter referred to as DBD; domain C); a variable hinge region (domain D); a conserved ligand-binding domain (hereinafter referred to as LBD; domain E); and a variable C-terminal region (domain F-Cell, supra).

The N-terminal region, which is highly variable in size and sequence, is poorly conserved among the different members of the superfamily. This part of the receptor is involved in the modulation of transcription activation.

The DBD consists of approximately 66 to 70 amino acids and is responsible for DNA-binding activity: This domain targets the receptor to specific DNA sequences, called hormone responsive elements (hereinafter referred to as HRE), within the transcription control unit of specific target genes on the chromatin. Steroid receptors such as GR, MR, PR, and AR recognize similar HRE DNA sequences, while the ER recognizes a different DNA HRE sequence. After binding to DNA, the steroid receptor is thought to interact with components of the basal transcriptional machinery and with sequence-specific transcription factors, thereby modulating the expression of specific target genes.

The LBD is located in the C-terminal part of the receptor and is primarily responsible for ligand binding activity. In this way, the LBD is essential for recognition and binding of the hormone ligand and, in addition, possesses a transcription activation function, thereby determining the specificity and selectivity of the hormone response of the receptor. Although moderately conserved in structure, the LBDs are known to vary considerably in homology among the individual members of the nuclear hormone receptor superfamily.

When a hormone ligand for a nuclear receptor enters the cell and is recognized by the LBD, it will bind to the specific receptor protein, thereby initiating an allosteric alteration of the receptor protein. (Cell, supra). As a result of this alteration the ligand/receptor complex switches to a transcriptionally active state and, as such, is able to bind through the presence of the DBD with high affinity to the corresponding HRE on the chromatin DNA. In this way, the ligand/receptor complex modulates expression of the specific target genes. The diversity achieved by this family of receptors results from their ability to respond to different ligands.

In addition to modulation of genomic activity, hormone receptor complexes can have important and varied non-genomic effects. This non-genomic activity is characterized by fast and transient activation of some important signaling pathways that affect cellular functions, such as proliferation and differentiation.

More generally, steroid hormone receptors are connected with embryonic development, adult homeostasis, and organ physiology. Various diseases and abnormalities are ascribed to a disturbance in steroid hormone action. Since steroid receptors exercise their influence as hormone-activated transcription modulators and as hormone-activated stimulators of non-genomic activity, further investigation into various approaches to modifying, interacting with, or modulating these receptors is an area of immense significance. For example, mutations and defects in these receptors, as well as over-stimulation or blocking of these receptors, may provide better insight into the underlying mechanism of the hormone signal transduction pathway, thereby leading to increased efficacy in treatment of a wide variety of steroid receptor-linked diseases and abnormalities.

Vitamin D3 and VDR and Cell Regulation

Vitamin D3 receptor (VDR) belongs to the nuclear hormone receptor superfamily. In the past few years, there has been a dramatic increase in evidence that supports the rapid, signaling action of various analogs of Vitamin D3 (Vit D3). Vit D3 has been shown to evoke transcellular movement of calcium across cellular membrane.

The classical signaling pathway of Vit D3 employs the VDR, which is a transcription factor for Vit D3 target genes. Effects of this pathway include inhibition of cellular growth and invasion. Cytoplasmic signaling pathways are increasingly being recognized, which similarly may regulate growth and differentiation but also apoptosis. Vit D3 inhibits the cell cycle at the G1/S checkpoint by upregulating the cyclin dependent kinase inhibitors p27 and p21, and by inhibiting cyclin D1. Indirect mechanisms include upregulation of TGF-β, and downregulation of the epidermal growth factor receptor.

Vit D3 may induce apoptosis either indirectly through effects on the insulin-like growth receptor and tumor necrosis factor-α or more directly via the Bcl-2 family system, the ceramide pathway, the death receptors (e.g. Fas) and the stress-activated protein kinase pathways (Jun N terminal kinase and p38).

Osteoporosis and other Bone Disorders. The development and homeostasis of bone is controlled largely by two different cells types: osteoblasts and osteoclasts. The bone matrix is secreted by osteoblasts, cells that lie on the surface of the existing bone matrix and deposit fresh layers of bone onto it. Mature osteoclasts are multinucleated cells of monocyte/macrophage origin that reabsorb calcified bone matrix. Ordinarily, the activities of these two cell types are tightly coordinated to maintain the structure and integrity of bone in an organism. However, the mechanisms that regulate the activities of these two cell types remain poorly understood and are largely unknown.

A number of diseases and disorders are associated with abnormal bone growth or abnormal increases or decreases in bone mass. For example, osteopetrosis is a thickening of the bone matrix and has been associated with defects in osteoclast maturation which make them unable to absorb bone (see, for example, Kong et al. Nature, 1999, 397:315-323; Soriano et al., Cell 1991, 64:693-702; Iotsova et al., Nat. Med. 1997, 3:1285-1289). By contrast, osteoporosis is a disease characterized by an increase in osteoclast activity, resulting in bones that are extremely porous, easily fractured, and slow to heal. Numerous other diseases and disorders that involve or are associated with abnormal bone growth and resorption are also known, including Paget's disease, osteogenesis imperfecta, fibrous dysplasia, hypophosphatasia, primary hyperparathyroidism, arthritis and periodontal disease to name a few. Additionally, osteolysis can be induced by many malignant tumors resident in or distant from bone, e.g., skeletal metastases in cancers of the breast, lung, prostate, thyroid, and kidney, humoral hypercalcemia during malignancy, and multiple myelomas.

Such diseases and disorders represent a major public health concern in the United States and in other countries. For example, it has been estimated that 10 million Americans, 80% of whom are women, are already afflicted with osteoporosis, while another 10 million individuals have low bone mass and are therefore at an increased risk for the disease.

There exists, therefore, a need for methods and compositions that can be used to identify cells such as osteoblast and/or osteoclast (for example in cell or tissue samples), and regulate or modulate the activities of such cells. There also exists a need for methods and compositions to treat diseases and disorders associated with abnormal bone growth and resorption, including the diseases discussed above, for example by modulating the activities of osteoblast and osteoclast cells. These and other needs in the art are addressed by the present invention.

Vit D3 and bone remodeling. In osteoblasts, treatment with Vit D3 leads to activation of Src, phospholipase C and formation of inositol triphosphate. Vit D3 augments the expression of TGF-β and the TGF-β receptor, both of which play an important role in the coupling of bone formation with resorption, and thereby the maintenance of bone mass. Vit D3 also interacts with Smad signaling system downstream from TGF-β receptor activation.

Vit D3 has an important impact on the local control of bone remodeling. It has antiproliferative and prodifferentiative actions on osteoblasts while playing a crucial role in bone resorption by stimulating the activity and formation of osteoclasts. Unlike osteoblasts, however, osteoclasts do not express the VDR. Therefore, Vit D3 regulates them indirectly, by controlling the expression of some cytokines and growth factors involved in the regulation of osteoclastogenesis and osteoclast function.

Vit D3 and Skin. The vitamin D receptor has been detected in most skin cells, which means that keratinization, hair growth, melanogenesis, fibrogenesis, angiogenesis, and immune-mediated processes are potential targets for vitamin D3.

Vit D3 has been shown to regulate melanocyte function by suppressing tyrosinase activity (necessary for melanogenesis, or melanin production) in normal melanocytes (Abdel-Malek et al., J Cell Physiol. 1988; 136(2):273-80). In addition, physiological concentrations of Vit D3 were shown to exhibit a growth inhibitory effect in primary melanocytes, and conferred resistance to several inductors of programmed cell death, including tumor necrosis factor-alpha and ultraviolet radiation (Sauer et al., Melanoma Res. 2003; 13(4): 339-47).

Vit D3 and Cancer. Mechanisms of inhibition of tumor invasion and metastasis potential which have been demonstrated include inhibition of serine proteinases, metalloproteinases and angiogenesis.

The lines of evidence for an effect of Vit D3 in systemic cancer are the demonstration of effects on cellular growth, differentiation, apoptosis, malignant cell invasion and metastasis; epidemiological findings of an association of the occurrence and outcome of cancers with derangements of vitamin D3 and its precursor; and the association of functional polymorphisms of the VDR with the occurrence of certain cancers. In addition, Vit D3 analogues are being developed as cancer chemotherapy agents.

There is accumulating evidence that the vitamin D3/1,25(OH)2D3/VDR axis is implicated in malignant melanoma. Melanoma cells express the VDR, and the antiproliferative and prodifferentiation effects of 1,25(OH)2D3 have been shown in cultured melanocytes, melanoma cells and melanoma xenografts. Recently, an inhibitory effect on the spread of melanoma cells has been demonstrated. In addition, low serum levels of 1,25(OH)2D3 have been reported in patients, and VDR polymorphisms have been shown to be associated with both the occurrence and outcome of malignant melanoma.

As in other cancers, there is evidence of a protective effect of Vit D3 in melanoma, but ultraviolet radiation, which is a principal source of Vit D3, is mutagenic.

Estrogen and ER and Cell Regulation

Estrogen (E2) exerts numerous biological effects in different tissues through an interaction with the ER. Amino acid sequence analyses, transient transfection studies, and mutational dissections of ER indicate that ER has the classical modular structure described above. The N-terminal A/B domain of the ER contains a transactivation function, referred to as transcriptional activation function 1 (TAF-1). The DBD contains two zinc fingers and is responsible for DNA recognition. The LBD and a second transactivation function, referred to as TAF-2, is located at the C-terminal of ER.

Upon binding to hormone, the ER undergoes an activation and transformation step. The activated ER interacts with specific estrogen response elements (EREs) that are located in the promoter region of estrogen-regulated genes and that influence its target gene transcription. Over the past decade, numerous studies have provided a basic understanding of both the effects of ligand (agonist/antagonist) on the ER and the relationship between the structure and function of the ER. Nevertheless, little is known regarding the mechanisms for the non-genomic activity of ER.

ERβ appears to be distinct from the more commonly known estrogen receptor, referred to as ERα. Collectively, ERα and ERβ are referred to herein as ER. The DBD of ERβ is 90% identical to that of ERα. However, the overall homology between the ligand binding domain (LBD) of ERα and ERβ is less than 55%. Like ER□, ER□ can stimulate transcription from an ERE in a ligand-dependent manner.

It has been established that estrogens induce fast and transient increases in the levels of intracellular second messengers, including calcium and cAMP, and that estrogens induce activation of mitogen-activated protein kinase (MAPK) and phospholipase C (Collins and Webb, 1999). In fact, numerous studies have demonstrated that estrogens induce rapid and transient activation of the Src/Ras/MAP kinase phosphorylation pathway. Activation of this pathway triggers vital cellular functions including cell proliferation and differentiation. The time course of these acute events parallels that elicited by peptide hormones, thus supporting the hypothesis that these events do not involve the ‘classical’ genomic action of estrogens.

Recent data also suggests a direct link between the estrogen receptor and the mitogen-activated protein (MAP) kinase-signaling cascade. MAP kinases are a family of serine-threonine kinases that are phosphorylated and activated in response to a variety of signals. These enzymes transduce extracellular signals from multiple membrane receptors to intracellular targets, including transcription factors, cytoskeletal proteins and enzymes. The MAP kinase family includes the extracellular-signal related kinases (ERKs), p38 and cJun N-terminal kinases which signal through a pathway involving sequential activation of Ras, Raf and mitogen-activated protein kinase (MEK). (S. M. Thomas, J. S. Brugge, Annual Review of Cell & Developmental Biology 13, 513-609, 1997). In pulmonary endothelial cells, neuronal cells, osteoblasts and osteoclasts, 17β-estradiol (E2) has been reported to rapidly activate the MAPK pathway, resulting in activities such as induction of acute dilation of blood vessels, neuroprotection in primary cortical neurons after glutamate excitotoxicity, and regulation of cell proliferation and differentiation in osteoclasts, leading to increased bone formation.

In breast cancer-derived cell lines, E2 activates the signal transducing Src/Ras/Erk pathway. The Src/Ras/ERk signaling pathway is a well-known target of growth factors. Importantly, activation of this pathway requires direct interaction of ER with Src. Activation of this pathway triggers different cellular responses such as proliferation or differentiation.

Androgens and Androgen Receptors and Cell Regulation

Androgen receptors (AR) are ligand-activated transcription factors that elicit highly selective, tissue-specific effects through regulation of divergent target genes.

Modulator Of Nongenomic Activity Of Estrogen Receptor (MNAR)

The present inventors have recently identified a novel scaffold protein, designated as MNAR (modulator of nongenomic activity of estrogen receptor). MNAR interacts with the estrogen receptor (ER) and this interaction is enhanced by 17β-estradiol (E2). MNAR controls ER interaction with members of the Src family of tyrosine kinases-p60^{src} (Src) and p56^{lck} (Lck). This interaction leads to stimulation of Src enzymatic activity and activation of MAP kinase pathway (Wong et al., Proc Natl Acad Sci USA 2003; 99: 14783-14788).

It has been demonstrated that MNAR interacts with Src via the SH3 domain of Src through a PXXP motif, and that the ER interacts with Src via the SH2 domain of Src. It has also recently been shown that MNAR mediates AR signaling similar to as for ER, via a ternary complex of AR/MNAR/Src (Unni et al., Cancer Res. 2004; 64: 7156-68.)

These data suggest that MNAR can potentially play a more general role in regulation of cellular processes. In addition to that, analysis of MNAR structure-functional organization indicates that MNAR is a scaffold protein that allows formation of multiple protein-protein contacts. Presence of several LXXLL motifs in the MNAR molecule indicates that MNAR can accommodate binding of multiple NR. In addition to interacting with ER using LXXLL, interactions with PR, GR and AR were also confirmed. In addition to that, presence of extended proline-rich motif and several classical PXXP motifs suggests potential interaction with proteins containing SH3 domains, which are present in multiple kinases and other signaling molecules. It has also recently been shown that MNAR interacts with SH3 domains of Src and p85-regulatory subunit of PI3 kinase.

It has been well established that nuclear receptors can regulate cellular processes using mechanism other than direct transcriptional regulation. ER, PR, AR and other nuclear receptors can activate multiple signaling cascades, which regulate expression of genes critically important for cell proliferation, differentiation and survival. Data presented previously, as well as analysis of MNAR functional organization, therefore suggest that MNAR could potentially be if not the main link, than at least one of the linker proteins that incorporates NR and other signaling molecules action in cellular communication cascades.

It is therefore reasonable to propose that MNAR can potentially represent an important target for development of functionally selective ligands of nuclear receptor ligands that can regulate important cellular functions.

It has previously been shown that VDR in the presence of Vit D3 interacts with cSrc. It also has been previously shown that Vit. D3 augments expression of the osteocalcin and alkaline phosphatase in cells of the osteoblastic lineage.

Accordingly, the present inventors undertook to evaluate the effect of MNAR overexpression on the expression of osteocalcin and alkaline phosphatase in osteosarcoma UMR 106 cells (ATCC CRL 1661 or ECACC 90111314) and ROS 17/2.8 cells. Considering that both osteocalcin and alkaline phosphatase are critical markers for osteoblasts differentiation, any regulation by MNAR would implicate MNAR as an important regulator of bone development.

### SUMMARY OF THE INVENTION

The present invention provides a novel interaction between protein modulator of non-genomic activity of nuclear receptors (MNAR) and the VitD3 nuclear hormone receptor (VDR). Specifically, it has been discovered that the VDR binds to the fifth “LXXLL” motif within the MNAR amino acid sequence. By this ligand-dependent interaction, the MNAR protein interacts with the VDR and activates MAP kinase signaling via formation of a ternary complex of VDR/MNAR/Src, leading to enhancement of VDR transcriptional activity.

In one embodiment, the present invention provides a method for identifying a ligand that modulates the interaction of MNAR with the VDR by adding a test compound to a reaction mixture of a MNAR polypeptide and a VDR polypeptide and evaluating the formation of a binding complex compared to the same reaction mixture in the absence of the test compound.

It is contemplated that the reaction mixture is cell-free or cell-based.

In a further embodiment, the method comprises detecting a complex between the VDR and the test compound.

The present invention also provides a method for identifying a ligand which modulates activity of the VDR in the presence of MNAR, by contacting a reaction mixture containing an MNAR polypeptide, and a host cell comprising a functional VDR, and detecting the activity of the VDR in the presence and absence of the test compound.

In one embodiment, the activity is phosphorylation of a MAP kinase.

In a specific embodiment, the kinase is Erk 1 and/or Erk 2.

In another embodiment, the activity is expression of a gene induced by activation of the VDR.

In a specific embodiment, the gene is osteocalcin or alkaline phosphatase.

The present invention also provides a method of modulating ligand-dependent activity in a cell expressing a functional VDR comprising contacting an MNAR polypeptide to the cell in the presence of a VDR ligand.

In one embodiment, the activity is phosphorylation of a MAP kinase.

In a specific embodiment, the kinase is Erk 1 and/or Erk 2.

In another embodiment, the activity is expression of a gene induced by activation of the VDR.

In a specific embodiment, the gene is osteocalcin or alkaline phosphatase.

### BRIEF DESCRIPTION OF THE DRAWINGS

FIG. 1. MNAR Interacts with the Ligand Binding Domain of VDR Full length MNAR was ³⁵S-radiolabeled by in vitro transcription/translation and incubated with GST-VDR ligand binding domain fusion protein in the presence or absence of 100 nM 1,25(OH)₂Vit D3. Bound material was isolated using glutathione-sepharose and analyzed by SDS-PAGE and autoradiography.

FIG. 2. VDR interacts with MNAR LXXLL #5. GST-VDR-LBD was incubated with vehicle or 1,25(OH)₂Vit.D3 at 100 nM concentration and then with immobilized peptides corresponding to different MNAR LXXLL motifs. VDR binding was detected using HRP-fused anti-GST antiserum in Wallac Victor² 1420 Multi label Counter. MNAR LXXLL motifs are numbered starting with the most N-terminal motif and designated as LXXLL 1-9, respectively.

FIG. 3. MNAR Enhances Vitamin D3 Stimulated Erk Activation UMR-106 cells transfected with control or MNAR expression plasmids were treated with 10 nM 1,25(OH)₂Vit.D3 for 0, 5, 10 or 20 minutes. Cells were harvested and the level of Erk 1 and 2 phosphorylation was determined using western blotting analysis.

FIG. 4. MNAR Modulates VDR Dependent Gene Expression A. Osteocalcin RNA levels were determined using TaqMan analysis from RNA isolated from UMR-106 cells transfected with control or MNAR expression plasmid and treated with vehicle or 100 nM 1,25(OH)₂Vit.D3 for 24 h. B. Alkaline Phosphatase RNA levels were determined using TaqMan analysis from RNA isolated from Ros 17/2.8 cells transfected with control or MNAR expression plasmid and treated with vehicle or 100 nM 1,25(OH)₂Vit.D3 for 48 h.

FIG. 5. This figure depicts the a schematic diagram of MNAR organization. The N-terminal portion of MNAR is termed the nuclear receptor interaction domain (NRID) due to the presence of multiple LXXLL motifs, while the C-terminal region of MNAR is called the proline and glutamic acid rich domain (PERD), due to the presence of many praline and glutamic acid residues. LXXLL and PXXP motifs are designated 1-10 and 1-2, respectively, starting from the most N-terminal motif.

### DETAILED DESCRIPTION

The present invention demonstrates that VDR MNAR and cSrc interact in a ligand-dependent manner. Direct interactions between VDR/MNAR and VDR/cSrc are mediated through the ligand binding domain of VDR, while the VDR/MNAR interaction requires MNAR's LXXLL motif number 5. In addition, the SH2 domain of cSrc is sufficient for the VDR/Src interaction, while the MNAR/Src interaction occurs via the SH3 domain of Src as previously demonstrated.

The present invention demonstrates that that MNAR interacts with VDR and forms a ternary complex with Src, and that MNAR overexpression strongly potentiates expression of the osteocalcin and alkaline phosphatase genes in UMR 106 and ROS 17/2.8 osteosarcoma cells. Since, as stated above, both osteocalcin and alkaline phosphatase are critical markers for osteoblasts differentiation, these data indicate that MNAR is an important regulator of bone development. Accordingly, the generation or identification of functionally selective ligands that modulate the MNAR interaction with the VDR, and hence, regulate osteocalcin and alkaline phosphatase expression will be useful for the treatment of osteoporosis.

These results provide additional evidence that MNAR is a scaffolding protein that incorporates nuclear receptors action into Src mediated cell signaling and are consistent with the model of MNAR action.

### Definitions

Vitamin D3, and its precursor, 1,25 dihydroxy vitamin D3 (Vit D3 and 1,25(OH)₂D3, respectively), is also known as cholecalciferol, (+)-5,7-cholestadien-3β-ol or Racumin D (CAS No. 67-97-0), and is necessary for the ultilization of calcium and phosphorus, by stimulating absorption, and for the assimilation of Vitamin A.

The human vitamin D3 receptor, or VDR, in a specific embodiment, contains a sequence as depicted in Swis Prot Database No. P11473 and GenBank Accession No. J03258. cDNA nucleotide sequences and amino acid sequences for the human VDR are shown in SEQ ID NOs: 1 and 2, respectively.

A “functional VDR” is a VDR expressed in a host cell that is capable of second messenger signaling, e.g., phosphorylation of kinases in the MAP kinase pathway such as Erk 1 and Erk 2, and/or is capable of regulating ligand-dependent gene transcription, e.g., of osteocalcin or alkaline phosphatase, or that is capable of eliciting any other activity which has been demonstrated upon being contacted with a natural or synthetic ligand.

A “VDR ligand” includes any natural ligand of the VDR, such as 1,25(OH)2D3, and any synthetic ligand or analogue. More than 3000 synthetic analogs of Vit D3 are known (Carlberg et al., Expert Opin. Ther. Patents 2003; 13(6): 761-72).

A “VDR ligand-binding domain” or VDR-LBD comprises, at a minimum, the C-terminal region of the amino acid sequence in SEQ ID NO: 2, or another ortholog of VDR. The VDR LBD sequence begins at residue 121 and continues to about residue 427. Preferably, the LBD encompasses residues 110-427.

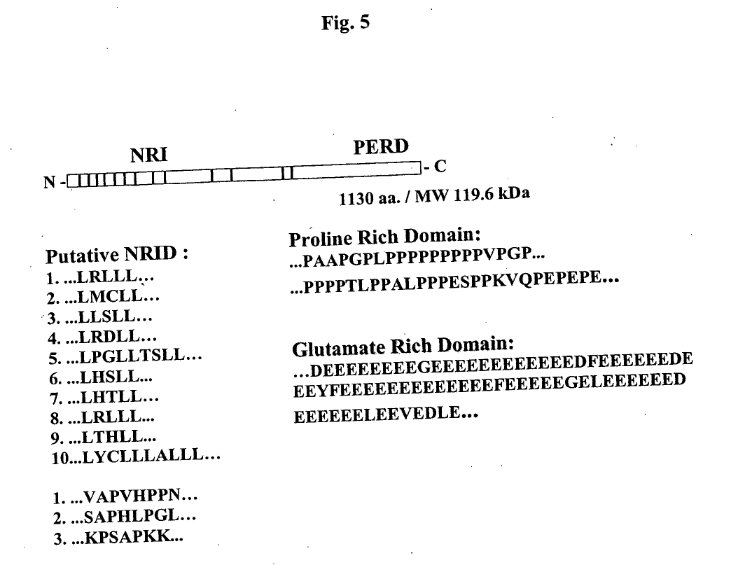

Modulator of nongenomic activity of estrogen receptor (MNAR) refers to the protein having a nucleotide and amino acid sequence depicted in GenBank Accession No. AF547989 (SEQ ID NOs: 3 and 4, respectively). As shown in FIG. 5, the LXXLL motifs are in the N-terminal region, and are numbered 1-10.

As used herein, a “ternary complex” refers to a complex which includes a nuclear hormone receptor, MNAR and Src or pI3 kinase (or other signaling kinase). In one embodiment, the ternary complex is the VDR/MNAR/Src complex or the VDR/MNAR/PI3 kinase complex. More specifically, the VDR/MNAR/Src complex is formed through an interaction of the ligand-binding domain of VDR with MNAR via LXXLL domain number 5 of MNAR, and with cSrc via the SH2 domain of cSrc; and the interaction of a PXXP motif of MNAR with the SH3 domain of cSrc. Additional ternary complexes include ER/MNAR/PI3 kinase and AR/MNAR/src.

The term “bone formation” is the process of bone synthesis and mineralization. The osteoblast cell modulates the process.

The term “bone growth” is the process of skeletal expansion. This process occurs by one of two ways: (1) intramembraneous bone formation arises directly from mesenchymal or bone marrow cells; (2) longitudinal or endochondral bone formation arises where bone from cartilage.

The term “osteogenesis” is synonymous with the term bone formation, defined above.

The terms “bone growth related disorder”, “bone growth associated disorder”, “bone growth disorder”, “bone growth disease” and other such variations thereof, as generally used herein, mean any disease or disorder related to the abnormal growth, repair development, resorption, degradation or homeostasis of bone tissue. Bone growth related disorders may therefore include diseases and disorders that are associated with abnormal increases, as well as abnormal decreases of bone mass in individuals. Also, the bone growth related disorders which are the subject of the present invention may include, but are not limited to, disorders that are associated with abnormal (e.g., increased or decreased) activity of osteoclast cells. The bone growth related disorders which are the subject of the present invention further include disorders that are associated with abnormal (e.g., increased or decreased) activity of osteoblast cells.

Exemplary bone growth related disorders that may be diagnosed or treated according to the methods and compositions of the present invention include osteopetrosis, osteoporosis, Paget's disease, osteogenesis imperfecta, fibrous dysplasia, hypophosphatasia, primary hyperparathyroidism arthritis, peridontal disease and myeloma blood diseases to name a few. Additionally, osteolysis can be induced by many malignant tumors resident in or distant from bone, e.g., skeletal metastases in cancers of the breast, lung, prostate, thyroid, and kidney, humoral hypercalcemia during malignancy, and multiple myelomas.

Human osteocalcin, also known as bone gla protein or BGP, has nucleic acid (cDNA) and amino acid sequences as depicted in GenBank Accession No. NM_{—}199173, and SEQ ID NOs: 5 and 6, respectively.

Alkaline phosphatase is well known in the pertinent art. As an example, the nucleic acid and amino acid sequences of the rat alkaline phosphatase, as found in the rat osteosarcoma cell lines described herein, can be found in GenBank Accession No. J03572, and are depicted in SEQ ID NOs: 7 and 8, respectively.

The term “label” refers to an entity that is directly detectable or that can bind to another molecule to permit detection. Direct labels include enzymes, fluorophores, chromosphores, radioisotopes, dyes, colloidal gold, colloidal carbon, latex particles, and chemiluminescent agents. Indirect labels include glutathione S-transferase (which binds glutathione and antibodies); FLAG, myc tag, which bind antibodies; His tag, which chelates nickel; biotin, which binds avidin; streptavidin, (or any of the three which bind to biotin); and Ig constant region domains, which bind antibodies and Fc molecules.

Labels can be introduced covalently, e.g., using conjugation chemistry as part of a fusion construct (as exemplified infra).

### Molecular Biology

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “molecule” means any distinct or distinguishable structural unit of matter comprising one or more atoms, and includes, for example, polypeptides and polynucleotides.

As used herein, the term “ligand” refers to molecules, including chemical compounds, peptides, and peptide mimetics, which bind to MNAR and VDR, and thereby alter or affect the function or behavior of cells expressing VDR, or prevent or alter the effect which another biologically active protein would otherwise have upon those cells. The term ligand and compound can be used interchangeable when referring to rational drug design, screening, and interaction assays.

A “test substance” or “test compound” or “test ligand” (including peptides and peptidomimetics) is a substance which has been identified or designed to interact with MNAR, preferably via the LXXLL motif of MNAR, whereby MNAR interaction with the VDR is also permitted.

A “lead compound” is a test compound which has been shown to bind to MNAR and modulate second messenger activity (e.g., transcriptional regulation) through the VDR upon interaction of MNAR and VDR.

Non-human animals include, without limitation, laboratory animals such as mice, rats, rabbits, hamsters, guinea pigs, etc.; domestic animals such as dogs and cats; and, farm animals such as sheep, goats, pigs, horses, and cows, and especially such animals made transgenic with human or murine MNAR and/or VDR.

In accordance with the present invention, there may be employed conventional molecular biology, microbiology and recombinant DNA techniques within the skill of the art. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

A “gene” is a sequence of nucleotides which code for a functional “gene product”. Generally, a gene product is a functional protein. However, a gene product can also be another type of molecule in a cell, such as an RNA (e.g., a tRNA or a rRNA). For the purposes of the present invention, a gene product also refers to an mRNA sequence which may be found in a cell. For example, measuring gene expression levels according to the invention may correspond to measuring mRNA levels. A gene may also comprise regulatory (i.e., non-coding) sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” and expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

A “promoter sequence” is a DNA regulatory region capable of binding RNA polymerase in a cell and initiation transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (e.g., a mRNA or a rRNA) or a protein. The expression product itself, e.g., the resulting RNA or protein, may also said to be “expressed” by the cell.

The term “transfection” means the introduction of a foreign nucleic acid into a cell. The term “transformation” means the introduction of a “foreign” (i.e., extrinsic or extracellular) gene, DNA or RNA sequence into a host cell so that the host cell will express the introduced gene or sequence to produce a desired substance, in this invention typically an RNA coded by the introduced gene or sequence, but also a protein or an enzyme coded by the introduced gene or sequence. The introduced gene or sequence may also be called a “cloned” or “foreign” gene or sequence, may include regulatory or control sequences (e.g., start, stop, promoter, signal, secretion or other sequences used by a cell's genetic machinery). The gene or sequence may include nonfunctional sequences or sequences with no known function. A host cell that receives and expresses introduced DNA or RNA has been “transformed” and is a “transformant” or a “clone”. The DNA or RNA introduced to a host cell can come from any source, including cells of the same genus or species as the host cell or cells of a different genus or species.

The terms “vector”, “cloning vector” and “expression vector” mean the vehicle by which a DNA or RNA sequence (e.g., a foreign gene) can be introduced into a host cell so as to transform the host and promote expression (e.g., transcription and translation) of the introduced sequence. Vectors may include plasmids, phages, viruses, etc. and are discussed in greater detail below.

A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A large number of vectors, including plasmid and fungal vectors, have been described for replication and/or expression in a variety of eukaryotic and prokaryotic hosts. The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal).

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, and mammalian host cells and vectors. For example, MNAR or VDR may be expressed in PC12, COS-1, or C2C12 cells. Other suitable cells include CHO cells, HeLa cells, 293T (human kidney cells), mouse primary myoblasts, and NIH 3T3 cells. In a preferred embodiment, the cells are osteosarcoma cells.

The term “heterologous” refers to a combination of elements not naturally occurring. For example, the present invention includes chimeric RNA molecules that comprise an rRNA sequence and a heterologous RNA sequence which is not part of the rRNA sequence. In this context, the heterologous RNA sequence refers to an RNA sequence that is not naturally located within the ribosomal RNA sequence. Alternatively, the heterologous RNA sequence may be naturally located within the ribosomal RNA sequence, but is found at a location in the rRNA sequence where it does not naturally occur. As another example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene that the one it is operatively associated with in nature.

The terms “mutant” and “mutation” mean any detectable change in genetic material, e.g., DNA, or any process, mechanism or result of such a change. This includes gene mutations, in which the structure (e.g., DNA sequence) of a gene is altered, any gene or DNA arising from any mutation process, and any expression product (e.g., RNA, protein or enzyme) expressed by a modified gene or DNA sequence. The term “variant” may also be used to indicate a modified or altered gene, DNA sequence, RNA, enzyme, cell, etc.; i.e., any kind of mutant. For example, the present invention relates to altered or “chimeric” RNA molecules that comprise an rRNA sequence that is altered by inserting a heterologous RNA sequence that is not naturally part of that sequence or is not naturally located at the position of that rRNA sequence. Such chimeric RNA sequences, as well as DNA and genes that encode them, are also referred to herein as “mutant” sequences.

In addition, variants include modified proteins or peptide fragments with altered chemical properties which demonstrates enhanced binding to a target, e.g., MNAR or VDR, which include certain commonly encountered amino acids which are not genetically encoded may be used which include, but are not limited to, [beta]-alanine (B-Ala) and other omega-amino acids such as 3-aminopropionic acid (Dap), 2,3-diaminopropionic acid (Dpr), 4-aminobutyric acid and so forth; α-aminoisobutyric acid (Aib); ω-aminohyxanoic acid (Aha); Δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (phg); cyclohexylalanine (Cha); norleucine (Nle); 2-naphythylalanine (2-Nal); 4-chlorophenylalanine (Phe(4-Cl)); 2-fluorophenylalanine (Phe(2-F)); 3-flurophenylalanine (Phe(3-F)); 4-fluorophenylalanine (Phe(4-F)); penicillamine (Pen); 1,2,3,4-tetrahydroisoquinoline-3-carboxylic acid (Tic); [beta]-2-thienylalanine (Thi); methionine sulfoxide (MOS); homoarginine (hArg); N-acetyl lysine (AcLys); 2,3-diaminobutyric acid (Dab); 2,3-diaminobutyric acid (Dbu); p-aminophenylalanine (Phe(pNH2)); N-methyl valine (MeVal); homocysteine (hCys) and homoserine (hSer).

The terms “array” and “microarray” are used interchangeably and refer generally to any ordered arrangement (e.g., on a surface or substrate) or different molecules, referred to herein as “probes”. Each different probe of an arrays specifically recognizes and/or binds to a particular molecule, which is referred to herein as its “target”. Microarrays are therefore useful for simultaneously detecting the presence or absence of a plurality of different target molecules, e.g., in a sample. In preferred embodiments, arrays used in the present invention are “addressable arrays” where each different probe is associated with a particular “address”. For example, in preferred embodiments where the probes of are immobilized on a surface or a substrate, each different probe of the addressable array may be immobilized at a particular, known location on the surface or substrate. The presence or absence of that probe's target molecule in a sample may therefore be readily determined by simply determining whether a target has bound to that particular location on the surface or substrate.

A nucleic acid molecule is “hybridizable” to another nucleic acid molecule (for example cDNA, genomic DNA, or RNA) when a single stranded form of the nucleic acid molecule can anneal to the other nucleic acid molecule under appropriate conditions of temperature and solution ionic strength (see, e.g., Sambrook et al., supra). The conditions of temperature and ionic strength determine the “stringency” of the hybridization. “Hybridization” requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences the greater the value of Tₘ for hybrids of nucleic acids having those sequences.

For hybrids of greater than 100 nucleotides in length, equations for calculating Tₘ having been derived (see, Sambrook et al., supra, 9.50-9.51).

In a specific embodiment, the term “standard hybridization conditions” refers to a Tₘ of about 55° C. and utilizes conditions as set forth above. In a preferred embodiment, the Tₘ is 60° C.; in a more preferred embodiment, the Tₘ is 65° C. In a specific embodiment, the term “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Low stringency hybridization conditions corresponding to a melting temperature of about 55° C. can be used (for example, 5×SSC, 0.1% SDS, 0.25% milk and no formamide; or, alternatively, 30% formamide, 5×SSC, and 0.5% SDS). Moderate stringency hybridization conditions correspond to a higher Tₘ., e.g., 40% formamide with 5× or 6×SSC. High stringency hybridization conditions correspond to the highest Tₘ, e.g., 50% formamide, 5× or 6×SSC. A 1×SSC solution is understood to be a solution containing 0.15 M NaCl and 0.015 M Na-citrate.

### Applications and Uses

The present invention relates to the discovery that MNAR interacts with the VDR, thereby mediating ligand binding to VDR. MNAR interacts with VDR specifically via the fifth LXXLL motif in the MNAR amino acid sequence (SEQ ID NO. 2). Accordingly, ligands which bind to this motif and thereby agonize or antagonize VDR binding to MNAR, are contemplated for use for modulating signaling through the VDR, e.g., through cSrc or PI3 kinase.

Using methods described herein, it is also possible to identify lead compounds or peptides, or test rationally designed compounds or peptides, which bind to or otherwise interact with MNAR and VDR. For example, Vit D3 can be used to antagonize binding of a test compound to a reaction comprising MNAR-VDR in order to determine, whether, in the absence of Vit D3, the test compound can substitute and mediate MNAR-VDR interaction and/or activity.

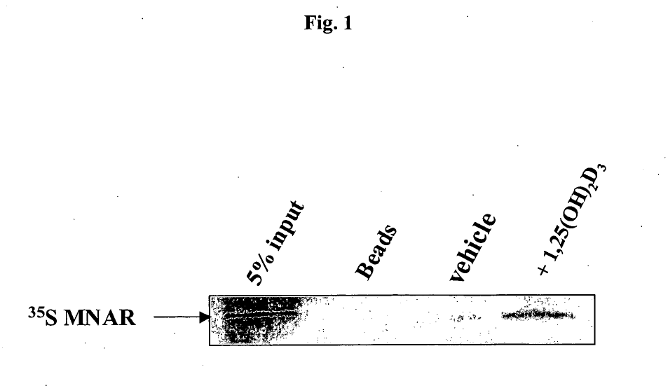

At a minimum, the methods of the present invention depend upon LXXLL motif number 5 in MNAR (see FIG. 1), amino acids 110-427 of VDR, which encompasses the VDR LBD and binds to MNAR.

### Interaction and Activity Assays

Interaction assays. The agonists or antagonists of MNAR function could act by either modulating an interaction between MNAR and VDR or other nuclear steroid hormone receptor, or MNAR and cSrc or PI3 kinase or another kinase, or by modulating an activity of MNAR or a VDR or other steroid hormone receptor. Once can employ any of the well-known two-hybrid assays or other conventional assays to study protein-protein interactions and the disruption thereof by test compounds. In vitro systems can be readily designed to identify lead ligands capable of specifically binding the MNAR according to present invention. Generally, such screening assays involve preparation of a reaction mixture, comprising a wild-type MNAR protein and a test compound, under conditions and for a time sufficient to allow the two compounds to interact (e.g., bind), thereby forming a complex that may be detected. The assays may be conducted in any of a variety of different ways including using microarrays.

Protein binding assays and gel shift assays are useful approaches to detect binding. Exemplary assays include assaying labeled VDR or cSrc binding to immobilized MNAR, and labeled MNAR or MNAR peptide binding to immobilized nuclear receptor (e.g., VDR) or modulator, e.g., cSrc. Many appropriate assays are amenable to scaled-up, high throughput usage suitable for volume drug screening. The particular assay used will be determined by the particular nature of the MNAR interactions. Assays may employ a single MNAR, MNAR fragments, MNAR fusion products, partial MNAR complexes, or the complete basal transcription complex comprising an MNAR nucleic acid.

Detection of MNAR-VDR-kinase complexes may be achieved using specific binding assays such as immunoassays, and biotin/avidin (including streptavidin and neutravidin) binding interactions. Commercial antibodies to VDR and kinases such as cSrc, various labels such as myc and FLAG tags, and the second messenger kinases that can be used to practice the invention are available from several sources, including R&D Systems, Minneapolis, Minn. and Santa Cruz Biotechnology, Santa Cruz, Calif. Antibodies to MNAR have been described in published in PCT Application WO2004/031223, incorporated herein by reference in its entirety. Such immunoassays include radioimmunoassay, ELISA (enzyme-linked immunosorbant assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

Other assays, including but not limited to, two-hybrid assays and surface plasmon resonance (SPR) assays, could be used to identify such interactions. Yeast two-hybrid assays as described by Young and Ozenberger in U.S. Pat. No. 5,989,808, which is herein incorporated by reference. SPR is described in Wegner et al., Anal. Chem. 2002; 74: 5161-5168; Cooper et al., Anal Bioanal Chem. 2003;377(5):834-42; and Gambari, Curr Med Chem Anti-Canc Agents. 200; 1(3):277-91).

Microarrays. The terms “array” and “microarray” are used interchangeably and refer generally to any ordered arrangement (e.g., on a surface or substrate) or different molecules, referred to herein as “probes”. Each different probe of an array specifically recognizes and/or binds to a particular molecule, which is referred to herein as its “target”. Microarrays are therefore useful for simultaneously detecting the presence or absence of a plurality of different target molecules, e.g., in a sample. The presence or absence of that probe's target molecule in a sample may therefore be readily determined by simply analyzing whether a target has bound to that particular location on the surface or substrate.

Conventional mircroarrays generally comprise a solid non-porous substrate, such as glass slide or a computer chip. In a typical microarray application, the substrate is contacted with a sample containing biomaterials to be analyzed. The substrate is then contacted with probe molecules such as labeled nucleic acids or polypeptides or other molecules. The labeled molecules bind with the molecules in the sample. The unbound probe molecules are removed, for example, by washing, and the microarray is then read by a suitable signal detection device, for example, by fluorescence emission.

For example, one embodiment comprises anchoring a protein, e.g., MNAR, or a test ligand, e.g., VDR or cSrc, onto a solid phase and detecting complexes of the protein and the test ligand that are on the solid phase at the end of the reaction and after removing (e.g., by washing) unbound ligands. For example, in one preferred embodiment of such a method, a protein may be anchored onto a solid surface and a labeled compound or polypeptide is contacted to the surface. After incubating the test ligand for a sufficient time and under sufficient conditions that a complex may form between the protein and the test compound or polypeptide, unbound molecules of the test ligand are removed from the surface (e.g., by washing) and labeled molecules which remain are detected.

In one embodiment, a mutant or variant protein may be anchored to a solid surface or support. Another labeled protein (the test compound), which may bind to the protein anchored to the solid surface, may be treated with a proteolytic enzyme, and its fragments may be allowed to interact with the protein attached to the solid surface. After washing, short, labeled peptide fragments of the treated protein may remain associated with the anchored protein. These peptides can be isolated and the region of the full length protein from which they are derived may be identified by the amino acid sequence.

In another, alternative embodiment, molecules of one or more different test compounds are attached to the solid phase and molecules of a protein (for example, a labeled MNAR polypeptide) may be contacted thereto. In such an embodiment, the molecules of different test compounds are preferably attached to the solid phase at a particular location on the solid phase so that test compounds that bind to a mutant protein may be identified by determining the location of bound proteins on the solid phase or surface. Again, mutant and variant proteins may be used as test compounds.

In addition, screening can be done using Vit D3 as an antagonist to a well containing an MNAR and VDR polypeptide, to which a test compound is added to the well in the presence and absence of Vit D3. Any binding and formation of a test compound/MNAR/VDR interaction should be inhibited by addition of unlabeled Vit D3.

Automated multiwell formats are the best developed high-throughput screening systems. Automated 96-well plate-based screening systems are the most widely used. The current trend in plate based screening systems is to reduce the volume of the reaction wells further, thereby increasing the density of the wells per plate (96-well to 384-, and 1536-well per plate). The reduction in reaction volumes results in increased throughput, dramatically decreased bioreagent costs, and a decrease in the number of plates which need to be managed by automation. For a description of protein arrays that can be used for high-throughput screening, see U.S. Pat. Nos. 6,475,809, 6,406,921, by 6,197,559, herein incorporated by reference.

Activity assays. Once a lead compound or compounds that specifically bind to an MNAR protein has been designed or identified and characterized, it can be used in assays to determine if it modulates signaling through the VDR. Examples of assays are described in the below and in the Examples.

Activity assays are generally designed to measure the activity of a target protein in the presence or absence of a test agent. Activity assays, including but not limited to mammalian transfection assays in which VDR transcriptional activity is observed, could be used to identify lead ligands. For example, ligands could be screened for their ability to modulate the enhancement of VDR transcriptional activity by MNAR. Alternatively, the ligands could be screened for their ability to affect second messenger signaling, such as Src, phospholipase C, Erk kinases 1 and 2. Such compounds may modulate the interactions of MNAR with VDR or they may modulate a known or unknown activity of MNAR or VDR including a transcriptional activity, e.g., of osteocalcin or alkaline phosphatase, or activation or enzymatic activity of kinases, such as e.g., phosphorylation.

One method used for screening for a ligand that modulates the activity of MNAR with VDR, comprises the steps of (a) contacting a test compound with an MNAR polypeptide which comprises the LXXLL motif capable of binding to VDR; and (b) determining whether said test compound specifically binds said polypeptide. In addition, the method can comprise the steps of (a) adding a test compound to a cell comprising the MNAR polypeptide and the VDR receptor; and (b) comparing the MNAR activity before and after the addition of the compound. Further approaches to this method involve adding a test compound to a control sample comprising a mutant cell which lacks MNAR activity or with significantly reduced MNAR activity.

There are numerous approaches for identifying compounds that affect non-genomic activity or genomic activity of nuclear receptors. In one embodiment, this comprises the steps of (a) adding a test compound to a cell comprising an MNAR and VDR complex; and (b) comparing the genomic versus non-genomic activity before and after. A selective genomic activity can be measured by conventional means. Preferably, the increase or positive effect of non-genomic activity, as measured, is a two-fold increase in, e.g. transcription, after addition of said test compound to a cell in the presence of MNAR and VDR when compared to genomic activity with test compound in the absence of MNAR, and wherein no change is observed in non-genomic activity after addition of said test compound. In these methods, preferably, at least one ligand of a nuclear receptor is present in the cell, or a kinase activated by such ligand is present, or both are present.

Alternatively, when selecting for test compounds for non-genomic activity, there should be at least about a two-fold increase in e.g., transcription, after addition of said test compound to a cell in the presence of MNAR-VDR when compared to non-genomic activity with test compound in the absence of MNAR and wherein no change is observed in genomic activity after addition of said test compound. One can measure the effect by determining an increase or decrease in the transcriptional activity of VDR in the presence of MNAR.

In each of these methods a control to assess the non-genomic activity (or genomic activity) of a compound is used, which control comprises administering a compound to a cell in the presence of MNAR and the VDR, and then repeating the experiment in the absence of MNAR and comparing the level of non-genomic activity, e.g., kinase activity. Preferred methods employ a cell which overexpresses MNAR or VDR and/or cSrc or PI3 kinase, or two or all three. The genomic activity of a compound is detected by having the VDR operatively associated with a reporter gene.

One way to detect second messenger activity is through kinase assays. This can be achieved using cell-free, cell-based, or animal model-based assays. Such assays can be used as secondary screens for the activity of candidate compounds selected in a primary screen. Kinase assays detect the phosphorlation state of kinases, typically by incorporating ³²P-into the cell, isolating cellular proteins, and immunoblotting with an antibody specific for phosphorus. Such assays are well known in the art.

Assays to detect changes, e.g., increases or decreases in transcription of a gene into e.g., mRNA are well-known in the art. Such assays include RT-PCR, including quantitative RT-PCR, Northern hybridization, transfection assays of the gene-of-interest linked to a reporter gene, etc.

Optimization

Once one or more drug leads to a target have been generated or identified, it is still often necessary to optimize a lead in order to improve its pharmacological efficacy. In this step, referred to as lead optimization, synthetic chemists chemically modify the lead in order to increase or decrease its binding to the target, modify its susceptibility to degradative pathways, or modify its pharmacokinetics.

Rational lead optimization employs theoretical methods to determine a set of likely lead candidates to a given target before experimentally screening lead-target binding. Rational lead optimization offers the possibility of reducing drug discovery time and expenses by reducing the number of potential lead-target screens.

Rational Drug Design and Screening for VDR Agonists/Antagonists Mediated by MNAR

Drug discovery occurs in two steps: 1) target identification and 2) lead identification and optimization. In the first step of target identification, a large molecule, which may be but is not limited to a cell-surface receptor or intra-cellular protein, referred to as the target, may be identified with a particular biological pathway or structure of interest. Once a potential target has been identified, it must be screened against a large number of small molecules to determine whether the target is appropriate for small molecule binding and interacting.

An MNAR-VDR interaction model can be used to create ligands of VDR, mediated by MNAR, which e.g., do not affect VDR-mediated transcription, but regulate VDR-mediated cell signaling, including e.g., activation of the MAP kinase pathway and phospholipase C-gamma pathway (PLCγ).

Structural identification. Identification and screening of modulators is further facilitated by determining structural features of the protein, e.g. MNAR or the interaction of MNAR with VDR, using X-ray crystallography, neutron diffraction, nuclear magnetic resonance spectrometry, and other techniques for structure determination. These techniques provide for the rational design or identification of modulators, including agonists and antagonists and partial agonists and antagonists.

For designing small molecules or peptidomimetics, it is beneficial to obtain a three dimensional structure for the pharmacophore of one or more compounds peptides. The term “pharmacophore” refers to the collection of functional groups on a compound or peptidomimetic that are arranged in three-dimensional space in a manner complementary to the target protein, e.g., MNAR, and that are responsible for biological activity as a result of ligand binding to the target protein. Useful three-dimensional pharmacophore models are best derived from either crystallographic or nuclear magnetic resonance structures of the target, but can also be derived from homology models based on the structures of related targets or three-dimensional quantitative structure-activity relationships derived from a previously discovered series of active compounds.

X-ray crystallography techniques are well known and are within the routine skill in the art. For example, see Cantor & Schimmel, Biophysical Chemistry 1980 (Vols. I-III) W. H. Freeman and Company (particularly Chapters 1-13 in Vol. I, and Chapter 13 in Vol. II). See, also, Macromolecular Crystallography, Parts A-B (Carter & Sweet, Eds.) In: Methods Enzymol. 1997, Vols. 276-277; Jan Drenth, Principles of Protein X-Ray Crystallography (New York: Springer-Verlag, 1994).

The crystal structure of VDR bound to its natural ligand, is known (Rochel et al., Mol Cell. 2000;5(1):173-9). Similarly, structurally and functionally important amino acids of the agonistic conformation of VDR are known, and include amino acid residues H229, D232, E269, F279, and Y295 (Vaisanen et al., Mol Pharmacol. 2002;62(4):788-94). Accordingly, given the LXXLL motif involved in the binding of MNAR to Vit D3, coupled with the critical regions of VDR that must bind to a ligand for activation, rationally designed ligands can be designed to mimic selective aspects of these activities.

Alternatively, the three-dimensional structures of MNAR-VDR interactions may generally be determined using nuclear magnetic resonance (NMR) techniques that are well known in the art. NMR data acquisition is preferably carried out in aqueous systems that closely mimic physiological conditions to ensure that a relevant structure is obtained. Briefly, NMR techniques use the magnetic properties of certain atomic nuclei (such as ¹H, ¹³C, ¹⁵N and ³¹P), which have a magnetic moment or spin, to probe the chemical environment of such nuclei. The NMR data can be used to determine distances between atoms in the molecule, which can be used to derive a three-dimensional model or the molecule.

### Potential Ligands

According to the present invention, ligands include peptide ligands (which further include peptidomimetics that are linear or cyclic) or chemical compounds, which selectively mimic the biological activity of the biologically active protein or which selectively block or selectively agonize the activity of the biologically active protein, such as VDR interaction with MNAR.

Compounds. The structural domains that mediate interactions between molecules, e.g., VDR and MNAR, and which also confer biological activity on cells expressing VDR receptors (or VDR-like receptors), can be used to rationally design compound modulators. These structural domains, and other functional domains, which can modulate the activity of these structural domains, can all be modified through a variety of means, including but not limited to site-directed mutagenesis, in order to either augment or reduce the biological activity. The structure and topology of these domains can all be used as a basis for the rational design of pharmaceuticals (small molecule conventional drugs or novel carbohydrate, lipid, DNA/RNA or protein-based high molecular weight biological compounds) to modulate (increase or decrease) the activity of VDR, or the VDR-MNAR complex, and/or the activity of the VDR/MNAR/other protein complexes. For example, using structural prediction calculations, possibly in conjunction with spectroscopic data like nuclear magnetic resonance, circular dichroism, and other physical-chemical structural data, or crystallographic data, or both, one can generate molecular models for the structure of MNAR and or VDR. These models, in turn, are important for rational drug design.

For screening, classes of compounds that may be identified include, but are not limited to, small molecules (e.g., organic or inorganic molecules which are less than about 2 kD in molecular weight, are more preferably less than about 1 kD in molecular weight) as well as macromolecules (e.g., molecules greater than about 2 kD in molecular weight). Compounds identified by these screening assays preferably also include peptides and polypeptides. For example, soluble peptides, fusion peptides members of combinatorial libraries (such as ones described by Lam et al., Nature 1991, 354:82-84; and by Houghton et al., Nature 1991, 354:84-86); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell 1993, 72:767-778). In addition, libraries of high-purity small organic ligands and peptide agonists that have well-documented pharmacological activities are available from Sigma-Aldrich (LOPAC LIBRARY™ and LIGAND-SETS™. Also available from Sigma-Aldrich is an Aldrich Library of Rare Chemicals, which is a diverse library of more than 100,000 small-molecule compounds, including plant extracts and microbial cultures. Other compound libraries are available from Tripos (LeadQuest®), TimTech (includes targeted libraries for kinase modulators).

Other companies that supply compound libraries include the following: 3-Dimensional Pharmaceuticals, Inc.; Advanced ChemTech; Abinitio PharmaSciences; Albany Molecular; Aramed Inc.; Annovis, Inc. (formerly Bearsden Bio, Inc.); ASINEX; AVANT Immunotherapeutics; AXYS Pharmceuticals; Bachem; Bentley Pharmaceuticals; Bicoll Group; Biofor Inc.; BioProspect Australia Limited; Biosepra Inc.; Cadus Pharmaceutical Corp.; Cambridge Research Biochemicals; Cetek Corporation; Charybdis Technologies, Inc.; ChemBridge Corporation; ChemDiv, Inc.; ChemGenics Pharmaceuticals Inc.; ChemOvation Ltd.; ChemStar, Ltd.; Chrysalon; ComGenex, Inc.; Compugen Inc.; Cytokinetics; Dextra Laboratories Ltd.; Discovery Partners International Inc.; Discovery Technologies Ltd.; Diversa Corporation; Dovetail Technologies, Inc.; Drug Discovery Ltd.; ECM Pharma; Galilaeus Oy; Janssen Pharmaceutica; Jerini Bio Tools; J-Star Research; KOSAN Biosciences, Inc.; KP Pharmaceutical Technology, Inc.; Lexicon Genetics Inc.; Libris Discovery; MicroBotanica, Inc.; MicroChemistry Ltd.; MicroSource Discovery Systems, Inc.; Midwest Bio-tech Inc.; Molecular Design & Discovery; MorphoSys AG; Nanosyn, Inc.; Ontogen Corporation; Organix, Inc.; Pharmacopeia, Inc.; Pherin Pharmaceuticals; Phytera, Inc.; PTRL East, Inc.; REPLICor Inc.; RSP Amino Acid Analogues, Inc.; Sanofi-Synthelab Pharmaceuticals; Sequitur, Inc.; Signature BioScience Inc.; Spectrum Info Ltd.; Talon Cheminformatics Inc.; Telik, Inc.; Tera Biotechnology Corporation.; Tocris Cookson; Torrey Pines Institute for Molecular Studies; Trega Biosciences, Inc.; and WorldMolecules/MMD.

In addition, the Institute of Chemistry and Cell Biology (ICCB), maintained by Harvard Medical School, provides the following chemical libraries, including natural product libraries, for screening: Chem Bridge DiverSet E (16,320 compounds); Bionet 1 (4,800 compounds); CEREP (4,800 compounds); Maybridge 1 (8,800 compounds); Maybridge 2 (704 compounds); Peakdale 1 (2,816 compounds); Peakdale 2 (352 compounds); ChemDiv Combilab and International (28,864 compounds); Mixed Commercial Plate 1 (352 compounds); Mixed Commercial Plate 2 (320 compounds); Mixed Commercial Plate 3 (251 compounds); Mixed Commercial Plate 4 (331 compounds); ChemBridge Microformat (50,000 compounds); Commercial Diversity Set1 (5,056 compounds); NCI Collections: Structural Diversity Set, version 2 (1,900 compounds); Mechanistic Diversity Set (879 compounds); Open Collection 1 (90,000 compounds); Open Collection 2 (10,240 compounds); Known Bioactives Collections: NINDS Custom Collection (1,040 compounds); ICCB Bioactives 1 (489 compounds); SpecPlus Collection (960 compounds); ICCB Discretes Collections. The following ICCB compounds were collected individually from chemists at the ICCB, Harvard, and other collaborating institutions: ICCB1 (190 compounds); ICCB2 (352 compounds); ICCB3 (352 compounds); ICCB4 (352 compounds). Natural Product Extracts: NCI Marine Extracts (352 wells); Organic fractions—NCI Plant and Fungal Extracts (1,408 wells); Philippines Plant Extracts 1 (200 wells); ICCB-ICG Diversity Oriented Synthesis (DOS) Collections; DDS1 (DOS Diversity Set) (9600 wells).

In a preferred embodiment, compound or peptide libraries designed which are target-based. There are numerous techniques available for creating more focused compound libraries rather than large, diverse ones. Chemical Computing Group, Inc. (Montreal) has developed software with a new approach to high-throughput drug design. The company's method uses high-throughput screening (HTS) experimental data to create a probabilistic QSAR (Quantitative Structure Activity Relationship) model, which is subsequently used to select building blocks in a virtual combinatorial library. It is based on statistical estimation instead of the standard regression analysis.

In addition, ArQule,Inc. (Woburn, Mass.) also has integrated technologies to perform high-throughput, automated production of chemical compounds and to deliver these compounds of known structure and high purity in sufficient quantities for lead optimization. Its AMAP™ (Automated Molecular Assembly Plant) performs high-throughput chemical syntheses for each phase of compound discovery.

Similarly compounds are often provided on online databases or on CD-ROM's for selective “cherry picking” of compounds. See, e.g., AbInitio PharmaSciences; ActiMol; Aral Biosynthetics; ASDI Biosciences; Biotechnology Corporation of America; Chembridge; ChemDiv; Florida Center—Heterocyclic Compounds; Microsource/MSDI; NorthStar; Peakdale; Texas Retaining Group; Zelinsky Institute; Advanced ChemTech; Ambinter; AnalytiCon Discovery; Aurora Fine Chemicals; Biofocus; Bionet/Key; Comgenex; Key Organics; LaboTest; Polyphor; SPECS and Biospecs; and Bharavi Laboratories.

Peptides. Peptide libraries useful for screening are available from the following sources: American Peptide Co., Inc.; BIOMOL Research Laboratories Inc.; Cell Sciences Inc.; GenoMechanix, LLC; Phoenix Pharmaceuticals Inc.; United States Biological; Advanced ChemTech Inc.; AerBio Ltd.; Amphotech Ltd.; AnaSpec Inc.; ANAWA Trading SA; Biomar Diagnostic Systems GmbH; BioSource International Inc.; Dalton Chemical Laboratories Inc.; Enzyme Systems Products Inc.; Peptides International Inc.; Princeton BioMolecules Corp.; Protein Technologies Inc.; Sigma-RBI; Synpep Corp.; and Xaia Custom Peptides.

Another approach uses recombinant bacteriophage to produce large libraries. Using the “phage method” (Scott and Smith, Science 1990, 249:386-390; Cwirla, et al., Proc. Natl. Acad. Sci. USA 1990, 87:6378-6382; Devlin et al., Science 1990, 49:404-406), very large libraries can be constructed (106-10 8 chemical entities). A second approach uses primarily chemical methods, of which the Geysen method (Geysen et al., Molecular Immunology 1986, 23:709-715; Geysen et al. J. Immunol. Meth. 1987, 102:259-274; and the method of Fodor et al. (Science 1991, 251:767-773) are examples. Furka et al. (14th International Congress of Biochemistry 1988, Volume #5, Abstract FR:013; Furka, Int. J. Peptide Protein Res. 1991, 37:487-493), Houghton (U.S. Pat. No. 4,631,211) and Rutter et al. (U.S. Pat. No. 5,010,175) describe methods to produce a mixture of peptides that can be tested as agonists or antagonists. Phage display kits are available from e.g.Ph.D.™

Peptides can be rationally designed and generated by identifying specific linear and constrained discrete portions of a biologically active proteins involved in protein-protein interactions, e.g., MNAR and VDR. By identifying such specific and discrete portions, biologically active peptides can be constructed which mimic the biological activity of the biologically active protein or which block the activity of the biologically active protein, or which selectively activate the biologically active protein. Thus, biologically active peptides can be constructed which act as ligands that act on mammalian cells by binding to the receptor sites of those cells to alter or affect their function or behavior, or to prevent the binding of the natural biologically active protein to the cellular receptor, thereby preventing the biologically active protein from affecting the cell, or to selectively modulate the receptor.

Peptides can be synthesized by those having ordinary skill in the art using well known techniques and readily available starting materials. According to the invention, references to synthesizing or constructing peptides is herein construed to refer to the production of peptides similar in sequence or structure to the corresponding regions of MNAR and VDR identified to be implicated in protein-protein interactions. These peptides may be produced using any method known in the art, including, but not limited to, chemical synthesis as well as biological synthesis in an in vitro or in vivo in a eukaryotic or prokaryotic expression system. The peptides may consist of only corresponding regions or they may comprise the corresponding sequences and addition sequences.

Peptides of the invention may be biologically active as produced or may require modification in order to assume a three-dimensional conformation which is biologically active. Generally, the peptides are active as produced. However, some modifications may be necessary for activity and some modifications may be desirable to improve or alter activity.

Modifications which may be performed, using standard techniques, according to the invention include but are not limited to cyclization, disulfide bond formation, glycosylation, phosphorylation, or the addition or subtraction of amino acid residues including amino acid residues which serve to produce a useful three dimensional conformation via a chemical linkage which is not generally found in natural peptides and/or mimetics including but not limited to, those described in Freidinger et al., 1980, Science 210:656; Hinds et al., 1988, J. Chem. Soc. Chem. Comm. 1447; Kemp et al., 1984, J. Org. Chem. 49:2286; Kemp et al., 1985, J. Org. Chem. 50:5834; Kemp et al., 1988, Tetrahedron Lett. 29:5077; Jones et al., 1988, Tetrahedron Lett. 29:3853.

Additionally, modifications may be performed, using standard techniques, according to the invention to create dimers or oligomers of the loops or multi-looped structures.

Peptidomimetics. Peptidomimetics are compounds in which at least a portion of the MNAR is modified, such that the three dimensional structure of the peptidomimetic remains substantially the same as that of the relevant interacting regions of the mature MNAR protein, and the compound retains the ability to bind to and modulate VDR. In one embodiment, the peptidomimetic is designed to bind to the VDR in a ligand-independent manner. In another embodiment, the peptidomimetic, as a single molecule, mimics both the MNAR binding region (to VDR) and a region of a VDR natural or synthetic ligand, and thus modulates VDR in a manner similar to the MNAR-ligand-VDR interaction.

Peptidomimetics may be peptide analogues that are, themselves, cyclic peptides containing one or more substitutions or other modifications within the MNAR sequence. Alternatively, at least a portion of the sequence may be replaced with a nonpeptide structure, such that the three-dimensional structure of the MNAR is substantially retained. In other words, one, two or three amino acid residues within e.g., the LXXLL sequence may be replaced by a non-peptide structure for improved binding.

In addition, other peptide portions of MNAR may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of conditions such as cancer or osteoprorosis. It should be noted that peptidomimetics may or may not have similar two-dimensional chemical structures, but share common three-dimensional structural features and geometry. Each peptidomimetic may further have one or more unique additional binding elements. The present invention provides methods for identifying peptidomimetics. A variety of modifications of peptide modifications are known in the art and can be used to generate peptidomimetic compounds. See, for instance, International Patent Publication No. WO 01/53331. Such modifications can also be used in the present invention to generate peptidomimetic compounds, as well as the specific modifications described below.

All peptidomimetics will ideally have a three-dimensional structure that is substantially similar to a three-dimensional structure of the e.g., LXXLL motif of MNAR. In general, two three-dimensional structures are said to be substantially structurally similar to each other if their pharmacophore atomic coordinates have a root-mean square deviation (RMSD) less than or equal to 1 angstrom, as calculated using the Molecular Similarity module within the QUANTA program (QUANTA, available from Molecular Simulations Inc., San Diego, Calif.). All peptidomimetics have at least one low-energy three-dimensional structure that is substantially similar to at least one low-energy three-dimensional structure of MNAR.

### In Vivo Screening

In one embodiment of the invention, drugs that inhibit the activity or formation of protein-protein complexes, are identified, tested, or optimized for preventing e.g., osteoporesis, transgenic mice or knock-out mouse such as the osteoblast ablation mouse (Mizuno et al., Biochem Biophys Res Commun. 1998 29;247(3):610-5) or a mouse overexpressing soluble osteoclast differentiation factor (Mizuno et al., Bone Miner Metab. 2002;20(6):337-44). The knock-out or transgenic animal assay system are utilized to test for agents or drugs that reduce or inhibit osteoporosis or other bone disorders by modulating the expression or activity of drug target proteins such as MNAR, specifically, drugs that modulate interactions between MNAR and VDR and/or cSrc or PI3 kinase.

In another aspect of the present invention, MNAR transgenic or knock-out mice can be used to further elucidate the role of MNAR in bone disorders, and any other disorders associated with steroid hormone receptors that are found to interact with MNAR, or to elucidate other proteins of which MNAR may interact with and modulate their activity.

Transgenic animals for use in the present invention can be prepared by any method, including, but not limited to, modification of embryonic stem (ES) cells and heteronuclear injection into blast cells, and such methods are known in the art (see, e.g., Coffman, Semin. Nephrol. 17:404, 1997; Esther et al., Lab. Invest. 74:953, 1996; Heddle, Environ Mol Mutagen 32:110 4, 1998; Werner et al., Arzneimittelforschung 48:870 80, 1998; U.S. Pat. No. 4,736,866 (Leder and Steward); U.S. Pat. No. 4,870,009 (Evans et al.); U.S. Pat. No. 5,718,883 (Harlan and June); U.S. Pat. No. 5,614,396 (Bradley et al.); and U.S. Pat. No. 5,650,503 (Archibald et al.).

A “knockout mammal” is a mammal (e.g., mouse) that contains within its genome a specific gene that has been inactivated by the method of gene targeting (see, e.g., U.S. Pat. Nos. 5,777,195 and 5,616,491). A knockout mammal may be either a heterozygote knockout (i.e., one defective allele and one wild type allele) or a homozygous mutant.

A “knock-in” mammal is a mammal in which an endogenous gene is substituted with a heterologous gene (Roamer et al., New Biol. 1991;3:331). Preferably, the heterologous gene is “knocked-in” to a locus of interest, either the subject of evaluation (in which case the gene may be a reporter gene; see Elegant et al., Proc. Natl. Acad. Sci. USA; 95:11897, 1998) of expression or function of a homologous gene, thereby linking the heterologous gene expression to transcription from the appropriate promoter. This can be achieved by homologous recombination, transposon (Westphal and Leder, Curr Biol 1997;7:530), using mutant recombination sites (Araki et al., Nucleic Acids Res, 25:868; 1997) or PCR (Zhang and Henderson, Biotechniques 1998;25:784). For example, transgenic “knock-in” animals can be created in which MNAR gene is stably inserted into the genome of the transgenic animal; or where animals are genetically engineered to constitutively express endogenous MNAR, e.g., by gene activation technology.

Treatment of Bone Disorders, Skin Disorders, and Cancer

As used herein, the term “therapeutically effective amount” is meant to refer to an amount of a compound which produces a medicinal effect observed as reduction in the rate of bone loss in an individual when a therapeutically effective amount of a compound is administered to an individual who is susceptible to or suffering from a disease characterized by bone loss. Therapeutically effective amounts are typically determined by the effect they have compared to the effect observed when a composition which includes no active ingredient is administered to a similarly situated individual.

For example, ligands of VDR that are found to selectively stimulate the MAP kinase activity may possess important bone-sparing action, without affecting calcium homeostasis, which can be used for the treatment of osteoporosis. Alternatively, ligands of VDR that selectively regulate VDR-mediated cell signaling can be used to treat patients with skin cancers. This would be a significant improvement to treatment using Vit D3 since these drugs would not have the hypercalcemic effects of Vit D3. Lastly, the selective nature of the rationally designed ligands would reduce or abrogate the multitude of side effects often associated with Vit D3 treatment, such as hypercalcemia, which often results in nausea, headache, bone pain, high blood pressure, and kidney damage.

### EXAMPLES

The invention is also described by means of particular examples. However, the use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

The experiments below indicate that VDR ligand-dependently interacts with MNAR and that this interaction is mediated by MNAR LXXLL motif number 5. VDR, in the presence of Vit D3 interacts with cSrc (via the SH2 domain of cSrc), and MNAR overexpression augments Vit D3 induced activation of the MAP kinase pathway via MNAR-cSrc interaction (via the SH3 domain of src through a PXXP motif) and phosphorylation of Erk kinases 1 and 2.

Interaction of VDR1/MNAR/PI3 kinase is also expected based on results observed with ternary complex formation involving ER/MNAR/PI3 kinase, described below. It was found that, similar to cSrc, MNAR interacts with PI3 kinase via the SH3 domain of the p85 subunit of PI3 kinase.

These results provide additional evidence that MNAR is a scaffolding protein that incorporates nuclear receptor interaction into kinase-mediated cell signaling, and are consistent with the model of MNAR action was previously proposed.

### Example 1

### Association of MNAR with VDR

Methods

Reagents. 1,25(OH)₂ Vit D3 was obtained from Sigma. Biotinylated peptides corresponding to different MNAR LXXLL motifs were synthesized and purified by Peptide Chemistry group at Wyeth Research. Glutathione sepharose beads were obtained from Sigma. Anti-phosphotyrosine antibody, SuperSignal Elisa Pico peroxidase substrate and Reacti-Bind™ NeutrAvidin™ coated microplate were from Pierce.

GST pull-down interaction analysis. A GST fusion to the ligand binding domain of VDR, amino acids 110-427, termed GST-VDR-LBD, was expressed in BL-21 cells and bound to glutathione-Sepharose 4B (Amersham Biosciences, Piscataway, NJ). Wild type MNAR was transcribed/translated and ³⁵S radiolabeled using Promega TNT Quick Coupled Transcription/Translation System (Madison, Wis.) and incubated for 1 hour at room temperature with GST-VDR-LBD fusion protein bound to glutathione-Sepharose 4B in the absence or presence of 100 nM 1,25(OH)2 Vit. D3 in binding buffer (50 mM Tris pH 8.0, 150 mM NaCl, 1 mM DTT, 1 mM PMSF, 1 mM EDTA, 0.05% NP-40, 1× Protease cocktail). Beads were then washed 4 times with the binding buffer and bound proteins were eluted by addition of SDS buffer and analyzed by SDS-PAGE and autoradiography.

ELISA-based VDR-MNAR interaction analysis. A rapid, nonisotopic ELISA-type method was used for characterization of VDR-MNAR interactions. Biotinylated peptides corresponding to different MNAR's LXXLL motifs (designated 1-9, sequentially from the most N-terminal motif) were synthesized and immobilized on a Reacti-Bind™ NeutrAvidin™-coated microplate (Pierce Biotechnology, Rockford, Ill.). The microplate was washed twice with binding buffer (50 mM Tris-HCL, pH 8.0, 150 mM NaCl, 1 mM DTT, 1 mM EDTA, 0.01% NP-40, and 0.01% BSA). Peptides were diluted in 100 μl binding buffer to a final concentration of 50 μM and incubated with the Reacti-Bind™ NeutrAvidin™ coated microplate for 1 hour at room temperature, washed 4 times with the binding buffer. GST-VDR-LBD, preincubated with vehicle or 1 μM 1,25(OH)₂D3, was allowed to interact with the immobilized peptide corresponding to one of each of the MNAR LXXLL motifs for 2 hours at room temperature. The plate was washed 4 times with binding buffer and incubated with anti-GST antibodies fused to HRP (Sigma, St. Louis, Mo.) for 1 h in binding buffer and washed 4 more times. SuperSignal® ELISA Pico Chemiluminescent Substrate (Pierce Biotechnology, Rockford, Ill.) was used to detect antigen/antibody complex, signal was read using a Wallac Victor2 1420 Multilabel Counter (PerkinElmer Lifesciences, Boston, Mass.).

### Results

MNAR interacts with ligand-binding domain of VDR. GST pull-down demonstrates that MNAR and VDR interact. Data presented in FIG. 1 indicate that MNAR directly interacts with VDR-LBD in a ligand-dependent manner.

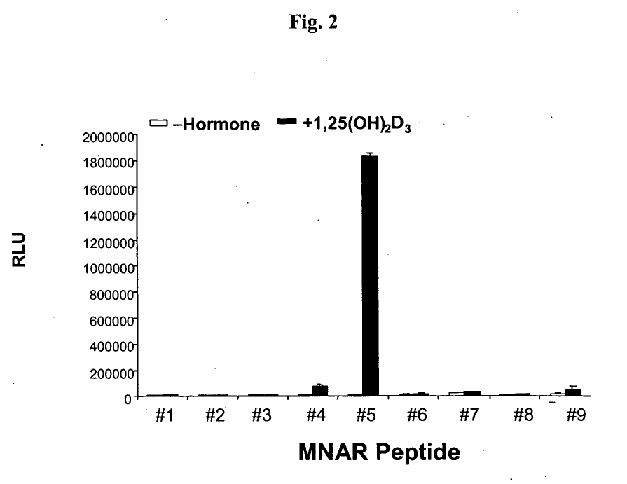

VDR Interacts with MNAR LXXLL motif #5. MNAR contains 10 putative LXXLL motifs that could potentially mediate MNAR interaction with nuclear hormone receptors. The results of the ELISA assay using anti-GST antibodies fused to HRP (FIG. 2) demonstrate that VDR ligand binding domain ligand-dependently interacted with the peptide corresponding to MNAR LXXLL motif #5.

### Example 2

### Agonist Activation of VDR Activity by MNAR

### Methods

Western blotting analysis. UMR-106 cells were transfected with control or MNAR expression vector using Lipofectamine 2000 reagent following the manufacturer's suggested procedures. Cells were cultured for an additional 48 hours following transfection in media supplemented with 2% charcoal stripped FBS. After 48 h cells were treated with 10 nM 1,25(OH)2D3 for the indicated time. Cells were rinsed and harvested in cold PBS, then centrifuged and supernatant was removed. Cells were lysed with 2 volumes of lysis buffer (20 mM Tris-HCL pH 7.5, 150 mM NaCl, 1 mM Na2EDTA, 1 mM EGTA, 1% Triton, 2.5 mM sodium pyrophosphate, 1 mM beta-glycerophosphate, 1 mM Na₂VO₄, 1 μg/μl leupeptin). Cell debris were removed by centrifugation and equal amount of protein was run on SDS-PAGE. Separated proteins were transferred to nitrocellulose membrane and levels of MNAR, ERK, and p-ERK were determined by western blotting analysis.

Quantitative PCR (TaqMan) analysis. UMR-106 and ROS 17/2.8 were transfected with control or MNAR expression vectors using Lipofectamine 2000 reagent following manufactures suggested procedures. Following transfection cells were cultured in media supplemented with 2% charcoal stripped FBS. 16 hours after the transfection cells were treated with 100 nM 1,25(OH)₂ Vit.D3 for 24 or 48 hours. Cells were lysed and total RNA was isolated using RNeasy Kit (Quiagen). Quantitative PCR (TaqMan) was performed to determine osteocalcin levels in UMR-106 cells and alkaline phosphatase levels in ROS 17/2.8 cells.

### Results

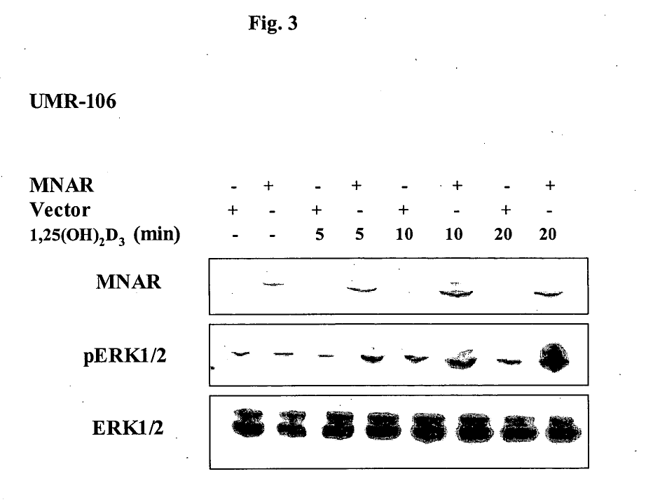

MNAR Enhances the Vitamin D Stimulation of Erk Activity. UMR-106 cells were transfected with control or MNAR expression vectors and treated with 10 nM 1,25(OH)₂ Vit. D3 for 0, 5, 10, or 20 min. The levels of Erk 1 and 2 phosphorylation in UMR-106 cells extracts were evaluated using western blotting analysis with antibody against phosphorylated Erk 1 and 2 (FIG. 3). Treatment of UMR-106 cells with 1,25(OH)₂ Vit D3 for 10 or 20 min resulted in augmentation of the levels of phosphorylated Erk 1 and 2 in the absence of MNAR. However, MNAR overexpression lead to dramatic increase in Erk phosphorylation. These results indicate that MNAR controls 1,25(OH)₂ Vit D3-induced activation of the MAP kinase pathway.

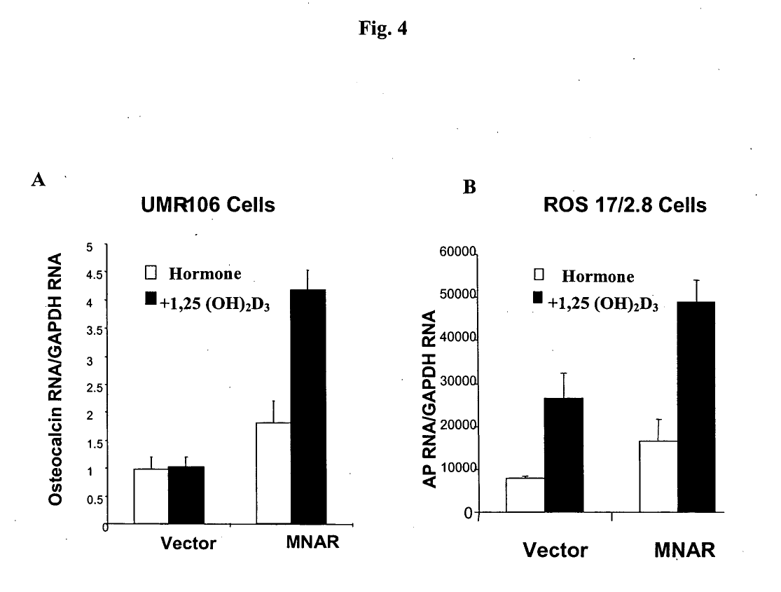

MNAR Modulates Vitamin D Dependent Gene Expression. Quantitative PCR was performed with RNA isolated from 1,25(OH)₂ Vit.D3-treated UMR 106 and Ros 17/2.8 cells to determine if MNAR can affect the 1,25(OH)₂ Vit D3 induced gene expression. UMR-106 cells were transfected with control or MNAR expression plasmids, RNA was isolated and analyzed using TaqMan analysis (FIG. 4A). Treatment with 1,25(OH)₂ Vit D3 itself did not affect the level of osteocalcin expression. However, MNAR overexpression resulted in strong potentiation of both basal and 1,25(OH)₂ Vit D3-stimulated levels of osteocalcin.

It was also determined whether MNAR affects expression of the alkaline phosphatase (AP)—an important marker for osteoblasts differentiation. RNA from vehicle or 1,25(OH)₂ Vit.D3 treated Ros 17/2.8 cells, transfected with control or MNAR expression plasmids, was isolated and used for TaqMan analysis (FIG. 4B). Overexpression of MNAR strongly enhanced the 1,25(OH)₂ Vit D3 induction of AP expression level. These results demonstrate that MNAR can regulate 1,25(OH)₂ Vit.D3-dependent gene expression and osteoblast cell differentiation and suggest that MNAR plays an important role in Vit D3 mediated bone development.

### Example 3

### Modulation of PI3 Kinase in ER by MNAR

### Methods

MNAR was recently shown to affect signaling mediated by the PI3/Akt kinase in ER via formation of an ER/MNAR/PI3 kinase ternary complex. It is expected that MNAR will have similar effects on PI3/Akt kinases which signal through the VDR.

Transfections and cell lysates preparation. MCF-7 cells were transfected with control, MNAR expression vector, non-specific siRNA or MNAR-specific siRNA using Lipofectamine 2000 reagent following manufactures suggested procedures. Cells were cultured for an additional 48 hours following transfection in media supplemented with 2% charcoal stripped FBS. After 48 hours, cells were treated with 10 nM 17β-estradiol for the indicated time. Cells were rinsed and harvested in cold PBS, then centrifuged and supernatant was removed. Cells were lysed with 2 volumes of lysis buffer (20 mM Tris-HCL pH 7.5, 150 mM NaCl, 1 mM Na2EDTA, 1 mM EGTA, 1% Triton, 2.5 mM sodium pyrophosphate, 1 mM beta-glycerophosphate, 1 mM NA₂VO₄, 1 μg/μl leupeptin). Cell debris was removed by centrifugation. For RNA-interference, non-specific siRNA or MNAR-specific siRNA were developed by Dharmacon Inc.

Immunoprecipitation and kinase assays. ERα was immunoprecipitated from cell lysates (1 mg/ml of protein) using the monoclonal anti-ERα antibody (D12, Santa Cruz) for 60 min at 4° C., then added with 20 μl of 50% suspension of protein G-Sepharose and incubated for an additional 60 min. The samples were centrifuged and pellets were washed with 1 ml of lysis buffer four times and analyzed by immunoblot. p85 was immunoprecipitated from cell lysates (1 mg/ml of protein) using anti-p85 polyclonal antibody (Upstate Biotech).

For PI3-K reaction, p85 was immunoprecipitated from cell lysates as described above. Kinase activity in the immunoprecipitate was detected using PI3-Kinase ELISA kit from Echelon Biosciences according to the manufacture's instructions. Briefly, reaction was run in 50 μl volume by adding 5 μl of 10× reaction buffer and 10 μl of PI(4,5)P₂ substrate solution (10 μM) to the p85 immunoprecipitate. After the PI3-K reactions was complete, reaction products are first mixed and incubated with a PI(3,4,5)P₃ detecting antibody, then added to the PI(3,4,5)P₃-coated microplate for competitive binding. A peroxidase-linked secondary detection reagent and colorirnetric detection was used to detect PI(3,4,5)P₃ detecting antibody binding to the plate. The colorimetric signal was inversely proportional to the amount of PI(3,4,5)P₃ produced by PI 3-K activity.

Akt kinase activity was detected in immunoprecipitate with anti-Akt antibody using kit from Cell Signaling (cat# 9840). Briefly, 1 mg of GSK-3 fusion protein was added to the Akt-immunoprecipitate as a substrate. Kinase reaction was run in a reaction buffer supplied by the manufacturer. The level of p-GSK-3 produced in the kinase reaction was analyzed by western blotting analysis.

Western blotting analysis. Equal amount of protein from cell lysates was run on SDS-PAGE. Separated proteins were transferred to nitrocellulose membrane and levels of Akt and p-Akt were determined by western blotting analysis. Akt antibody was from Cell Signaling (anti-Akt cat#9272, anti-p-Akt cat# 9271).

### Results

ERα, MNAR and p85 interact in MCF-7 cells. It has previously been shown that ER interacts with the p85 subunit of PI3 kinase (Migliaccio et al., J. Steroid Biochem. Mol. Biol. 2002;83(1-5):31-5). It was first evaluated whether endogenous ER, MNAR and the regulatory subunit of PI3-K-p85, interact in MCF7 cells. To address this question quiescent MCF-7 cells were unstimulated, or stimulated with 10 nM estradiol for 20 min. Cells were lysed with the lysis buffer and cell lysates were used for immunoprecipitated with either anti-ERα, or anti-p85α antibodies. Each immunoprecipitate was analyzed by immunoblot with anti-p85α, anti-ERα or anti-MNAR antibodies. Results show that treatment with estradiol triggered co-immunoprecipitation of MNAR, p85 and ERα. No association was detected in control immunoprecipitates. These data indicate that endogenous MNAR-ERα and p85 interact in MCF7 cells. Specifically, MNAR interacts with p85 via the SH3 domain of p85.

Role of MNAR in estradiol-dependant activation of PI3-K. To investigate the effect of MNAR on estradiol induced activation of PI3-K, MCF-7 cells were transfected with either MNAR-expressing plasmid or MNAR specific siRNA to down-regulate MNAR expression. 48 hours after transfections, cells were untreated or treated with 10 nM estradiol for 20 min, harvested and the level of PI3-K activity in cell extracts was evaluated. To evaluate the PI3 kinase activity, the production of PtdIns-3P by the p85 immunoprecipitates was measured. Results show that treatment of MCF-7 cells with estradiol lead to stimulation of the PI3-kinase activity. MNAR overexpression increased estrogen-dependant activation of PI3-K. In contrast, depletion of cells from MNAR, using MNAR specific siRNA, resulted in decreased level of PI 3-K activation by estradiol. In addition, the PI3-K inhibitor, LY294002, as well as the ER antagonist, ICI 182 780, blocked the effect of estradiol on PI3-K activity. These data indicate that interaction between MNAR-ER and p85 leads to activation of PI3 kinase.

MNAR modulates estradiol-dependant activation of Akt. It has been well established that activation of PI3 kinase leads to activation of Akt, and that Akt is the primary mediator of PI3-K-initiated signaling. By modulating activity of downstream targets, Akt promotes cell survival and cell cycle progression. To evaluate whether MNAR affects Akt phosphorylation and activation in response to estradiol, MCF-7 cells were transfected with either MNAR-expressing plasmid or MNAR specific siRNA. 48 hours after transfections, the cells were untreated or treated with 10 nM estradiol for 20 min. Cell extracts were then prepared and analyzed for the level of phosphorylated Akt using Western Blotting analysis. It was found that treatment of MCF-7 cells with estradiol increased the level of phospho-Akt. In addition to that, strong attenuation of Akt phosphorylation was observed in cells transfected with MNAR specific siRNA, but not in cells transfected with non-specific siRNA. These results are consistent with strong increase in phospho-Akt level detected in cells transfected with MNAR-expressing plasmid, comparing to cells transfected with empty vector. These data indicate that MNAR regulates level of Akt phosphorylation in response to estradiol.

To determine if increased Akt phosphorylation leads to its activation, MCF-7 cells were transfected either with empty vector or a plasmid for MNAR overexpression. 48 hours after transfection, the cells were untreated or treated with estradiol for 20 min, then harvested and Akt was immunoprecipitated with anti-Akt antibody. Kinase reaction was performed with precipitated Akt using a polypeptide substrate derived from GSK-3. It is well known that activated Akt phosphorylates GSK-3. Level of the GSK-3 phosphorylation was evaluated using Western Blotting analysis. Results of this experiment demonstrate that MNAR overexpression stimulates estradiol induced Akt activation.

Taken together, these data demonstrate that MNAR plays critical role in activation of PI3/Akt kinase pathway in response to estradiol. The same can be expected from PI3/Akt kinase signaling through VDR, via interaction and ternary complex formation of VDR/MNAR/p85.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method for identifying a ligand which modulates interaction of MNAR with the vitamin D3 receptor, which method comprises:
(a) contacting a test compound to a reaction mixture that comprises
(i) a MNAR polypeptide containing the fifth LXXLL motif; and
(ii) a vitamin D3 receptor polypeptide containing the ligand-binding domain,
wherein the reaction mixture conditions permit binding of the MNAR polypeptide to the vitamin D3 receptor polypeptide to form a binding complex;
(b) detecting levels of formation of the binding complex in the reaction mixture in the presence of the test compound; and
(c) comparing the level of the binding complex formed in the presence of the test compound to the level of binding complex formed in the absence of said test compound,
wherein a increase in the level of the binding complex formed in the presence of the test compound indicates that the test compound may be a lead compound.

2. The method of claim 1, wherein the MNAR polypeptide comprises the amino acid sequence set forth in SEQ ID NO: 4.

2. The method of claim 1, wherein the reaction mixture is cell-based.

3. The method of claim 1, wherein the reaction mixture is cell-free.

4. The method of claim 1, wherein the MNAR polypeptide comprises a minimum of LXXLL motif number 5.

5. The method of claim 1, wherein the method further comprises detecting binding of the test compound to the vitamin D3 receptor polypeptide.

6. A method for identifying a ligand which modulates the activity of a vitamin D3 receptor upon interaction of MNAR with the vitamin D3 receptor, which method comprises:
(a) contacting a test compound to a reaction mixture that comprises
(i) a MNAR polypeptide; and
(ii) a host cell comprising a functional vitamin D3 receptor polypeptide,
wherein the reaction mixture conditions permit binding of the MNAR polypeptide to the vitamin D3 receptor to form a binding complex;
(b) detecting activity of the vitamin D3 receptor in the reaction mixture in the presence of the test compound; and
(c) comparing the level of the activity in the presence of the test compound to the level of activity in the absence of said test compound,
wherein a increase in the level of the activity in the presence of the test compound indicates that the test compound may be a ligand which modulates the activity of VDR.

7. The method of claim 6, wherein the MNAR polypeptide comprises the amino acid sequence set forth in SEQ ID NO: 4.

8. The method of claim 6, wherein the MNAR polypeptide comprises LXXLL motif number 5.

9. The method of claim 6, wherein the method further comprises detecting binding of the test compound to the vitamin D3 receptor polypeptide.

10. The method of claim 6, wherein the host cell is an osteosarcoma cell.

11. The method of claim 10, wherein the osteosarcoma cell is a UMR 106 cell or a ROS 17/2.8 cell.

12. The method of claim 6, wherein the activity detected is phosphorylation of the Erk 1 or Erk 2 kinase.

13. The method of claim 6, wherein the activity detected is expression of a gene induced by activation of the vitamin D3 receptor.

14. The method of claim 13, wherein the gene is osteocalcin or alkaline phosphatase.

15. The method of claim 14, wherein the gene is osteocalcin having a nucleotide sequence set forth in SEQ ID NO: 5, or a nucleotide sequence which hybridizes to the nucleotide sequence set fort in SEQ. ID NO: 5.

16. The method of claim 14, wherein the gene is alkaline phosphatase having a nucleotide sequence set forth in SEQ ID NO: 7, or having a nucleotide sequence which hybridizes to the nucleotide sequence set fort in SEQ ID NO: 7.

17. A method of modulating VDR ligand-dependent activity in a cell, comprising contacting a MNAR polypeptide to a reaction mixture that comprises
(i) a host cell comprising a functional vitamin D3 receptor polypeptide; and
(ii) a vitamin D3 ligand
wherein the activity of the vitamin D3 receptor in the reaction mixture in the presence of the MNAR polypeptide is different compared to the activity of the vitamin D3 receptor in the absence of the MNAR polypeptide.

18. The method of claim 17, wherein the host cell is an osteosarcoma cell.

19. The method of claim 18, wherein the osteosarcoma cell is a UMR 106 cell or a ROS 17/2.8 cell.

20. The method of claim 17, wherein the activity detected is phosphorylation of the Erk 1 or Erk 2 kinase.

21. The method of claim 17, wherein the activity detected is increase of expression of a gene induced by activation of the vitamin D3 receptor.

22. The method of claim 21, wherein the gene is osteocalcin or alkaline phosphatase.

23. The method of claim 22, wherein the gene is osteocalcin having a nucleotide sequence set forth in SEQ ID NO: 5, or a nucleotide sequence which hybridizes to the nucleotide sequence set fort in SEQ ID NO: 5.

24. The method of claim 22, wherein the gene is alkaline phosphatase having a nucleotide sequence set forth in SEQ ID NO: 7, or having a nucleotide sequence which hybridizes to the nucleotide sequence set fort in SEQ ID NO: 7.

25. A peptide comprising the LXXLL motif number 5 of MNAR and a detectable label, wherein the peptide is a fragment of MNAR.

26. The peptide of claim 25, wherein the peptide is LPGLLTSLL.

27. The peptide of claim 25, wherein the label is biotin.

28. A composition comprising the components as recited in claim 1.

29. A method for identifying a ligand which modulates signaling through the VDR receptor, which method comprises:
(a) contacting a test compound to a reaction mixture that comprises
(i) a MNAR polypeptide containing the fifth LXXLL motif; and
(ii) a vitamin D3 receptor polypeptide containing the ligand-binding domain,
(iii) a functional cSrc or PI3 kinase;
wherein the reaction mixture conditions permit binding of the MNAR polypeptide to the vitamin D3 receptor polypeptide and to the cSrc or PI3 kinase to form a ternary complex;
(b) detecting levels of formation of the ternary complex in the reaction mixture in the presence of the test compound; and
(c) comparing the level of the ternary complex formed in the presence of the test compound to the level of binding complex formed in the absence of said test compound,
wherein a increase in the level of the ternary complex formed in the presence of the test compound indicates that the test compound may be a lead compound.

30. A method for identifying a ligand which modulates the activity of a vitamin D3 receptor upon interaction of MNAR with the vitamin D3 receptor, which method comprises:
(a) contacting a test compound to a reaction mixture that comprises
(i) a MNAR polypeptide; and
(ii) a host cell comprising a functional vitamin D3 receptor polypeptide and a functional cSrc or PI3 kinase
wherein the reaction mixture conditions permit binding of the MNAR polypeptide to the vitamin D3 receptor and to the cSrc or PI3 kinase to form a ternary complex;
(b) detecting activity of the vitamin D3 receptor in the reaction mixture in the presence of the test compound; and
(c) comparing the level of the activity in the presence of the test compound to the level of activity in the absence of said test compound,
wherein a increase in the level of the activity in the presence of the test compound indicates that the test compound may be a ligand which modulates the activity of VDR.

31. The method of claim 30, wherein the kinase is PI3 kinase.

32. The method of claim 31, wherein the activity detected is phosphorylation of the Akt kinase.


SEQUENCE LISTING (8 sequences)

SEQ ID NO: 1 - DNA, 4604 bp, Homo sapiens
ggaacagcttgtccacccgccggccggaccagaagcctttgggtctgaagtgtctgtgagacctcacagaagagcacccctgggctccacttacctgccccctgctccttcagggatggaggcaatggcggccagcacttccctgcctgaccctggagactttgaccggaacgtgccccggatctgtggggtgtgtggagaccgagccactggctttcacttcaatgctatgacctgtgaaggctgcaaaggcttcttcaggcgaagcatgaagcggaaggcactattcacctgccccttcaacggggactgccgcatcaccaaggacaaccgacgccactgccaggcctgccggctcaaacgctgtgtggacatcggcatgatgaaggagttcattctgacagatgaggaagtgcagaggaagcgggagatgatcctgaagcggaaggaggaggaggccttgaaggacagtctgcggcccaagctgtctgaggagcagcagcgcatcattgccatactgctggacgcccaccataagacctacgaccccacctactccgacttctgccagttccggcctccagttcgtgtgaatgatggtggagggagccatccttccaggcccaactccagacacactcccagcttctctggggactcctcctcctcctgctcagatcactgtatcacctcttcagacatgatggactcgtccagcttctccaatctggatctgagtgaagaagattcagatgacccttctgtgaccctagagctgtcccagctctccatgctgccccacctggctgacctggtcagttacagcatccaaaaggtcattggctttgctaagatgataccaggattcagagacctcacctctgaggaccagatcgtactgctgaagtcaagtgccattgaggtcatcatgttgcgctccaatgagtccttcaccatggacgacatgtcctggacctgtggcaaccaagactacaagtaccgcgtcagtgacgtgaccaaagccggacacagcctggagctgattgagcccctcatcaagttccaggtgggactgaagaagctgaacttgcatgaggaggagcatgtcctgctcatggccatctgcatcgtctccccagatcgtcctggggtgcaggacgccgcgctgattgaggccatccaggaccgcctgtccaacacactgcagacgtacatccgctgccgccacccgcccccgggcagccacctgctctatgccaagatgatccagaagctagccgacctgcgcagcctcaatgaggagcactccaagcagtaccgctgcctctccttccagcctgagtgcagcatgaagctaacgccccttgtgctcgaagtgtttggcaatgagatctcctgactaggacagcctgtgcggtgcctgggtggggctgctcctccagggccacgtgccaggcccggggctggcggctactcagcagccctcctcacccgtctggggttcagcccctcctctgccacctcccctatccacccagcccattctctctcctgtccaacctaacccctttcctgcgggcttttccccggtcccttgagacctcagccatgaggagttgctgtttgtttgacaaagaaacccaagtgggggcagagggcagaggctggaggcaggccttgcccagagatgcctccaccgctgcctaagtggctgctgactgatgttgagggaacagacaggagaaatgcatccattcctcagggacagagacacctgcacctccccccactgcaggccccgcttgtccagcgcctagtggggtctccctctcctgccttactcacgataaataatcggcccacagctcccaccccacccccttcagtgcccaccaacatcccattgccctggttatattctcacgggcagtagctgtggtgaggtgggttttcttcccatcactggagcaccaggcacgaacccacctgctgagagacccaaggaggaaaaacagacaaaaacagcctcacagaagaatatgacagctgtccctgtcaccaagctcacagttcctcgccctgggtctaaggggttggttgaggtggaagccctccttccacggatccatgtagcaggactgaattgtccccagtttgcagaaaagcacctgccgacctcgtcctccccctgccagtgccttacctcctgcccaggagagccagccctccctgtcctcctcggatcaccgagagtagccgagagcctgctcccccaccccctccccaggggagagggtctggagaagcagtgagccgcatcttctccatctggcagggtgggatggaggagaagaattttcagaccccagcggctgagtcatgatctccctgccgcctcaatgtggttgcaaggccgctgttcaccacagggctaagagctaggctgccgcaccccagagtgtgggaagggagagcggggcagtctcgggtggctagtcagagagagtgtttgggggttccgtgatgtagggtaaggtgccttcttattctcactccaccacccaaaagtcaaaaggtgcctgtgaggcaggggcggagtgatacaacttcaagtgcatgctctctgcaggtcgagcccagcccagctggtgggaagcgtctgtccgtttactccaaggtgggtctttgtgagagtgagctgtaggtgtgcgggaccggtacagaaaggcgttcttcgaggtggatcacagaggcttcttcagatcaatgcttgagtttggaatcggccgcattccctgagtcaccaggaatgttaaagtcagtgggaacgtgactgccccaactcctggaagctgtgtccttgcacctgcatccgtagttccctgaaaacccagagaggaatcagacttcacactgcaagagccttggtgtccacctggccccatgtctctcagaattcttcaggtggaaaaacatctgaaagccacgttccttactgcagaatagcatatatatcgcttaatcttaaatttattagatatgagttgttttcagactcagactccatttgtattatagtctaatatacagggtagcaggtaccactgatttggagatatttatggggggagaacttacattgtgaaacttctgtacattaattattattgctgttgttattttacaagggtctagggagagacccttgtttgattttagctgcagaactgtattggtccagcttgctcttcagtgggagaaaaacacttgtaagttgctaaacgagtcaatcccctcattcaggaaaactgacagaggagggcgtgactcacccaagccatatataactagctagaagtgggccaggacaggccgggcgcggtggctcacgcctgtaatcccagcagtttgggaggtcgaggtaggtggatcacctgaggtcgggagttcgagaccaacctgaccaacatggagaaaccctgtctctattaaaaatacaaaaaaaaaaaaaaaaaaaaatagccgggcatggtggcgcaagcctgtaatcccagctactcaggaggctgaggcagaagaattgaacccaggaggtggaggttgcagtgagctgagatcgtgccgttactctccaacctggacaacaagagcgaaactccgtcttagaagtggaccaggacaggaccagattttggagtcatggtccggtgtccttttcactacaccatgtttgagctcagacccccactctcattccccaggtggctgacccagtccctgggggaagccctggatttcagaaagagccaagtctggatctgggaccctttccttccttccctggcttgtaactccaccaagcccatcagaaggagaaggaaggagactcacctctgcctcaatgtgaatcagaccctaccccaccacgatgtgccctggctgctgggctctccacctcaggccttggataatgctgttgcctcatctataacatgcatttgtctttgtaatgtcaccaccttcccagctctccctctggccctgcttcttcggggaactcctgaaatatcagttactcagccctgggccccaccacctaggccactcctccaaaggaagtctaggagctgggaggaaaagaaaagaggggaaaatgagtttttatggggctgaacggggagaaaaggtcatcatcgattctactttagaatgagagtgtgaaatagacatttgtaaatgtaaaacttttaaggtatatcattataactgaaggagaaggtgccccaaaatgcaagattttccacaagattcccagagacaggaaaatcctctggctggctaactggaagcatgtaggagaatccaagcgaggtcaacagagaaggcaggaatgtgtggcagatttagtgaaagctagagatatggcagcgaaaggatgtaaacagtgcctgctgaatgatttccaaagagaaaaaaagtttgccagaagtttgtcaagtcaaccaatgtagaaagctttgcttatggtaataaaaatggctcatacttatatagcacttactttgtttgcaagtactgctgtaaataaatgctttatgcaaacc

SEQ ID NO: 2 - protein, 427 aa, Homo sapiens
MEAMAASTSLPDPGDFDRNVPRICGVCGDRATGFHFNAMTCEGCKGFFRRSMKRKALFTCPFNGDCRITKDNRRHCQACRLKRCVDIGMMKEFILTDEEVQRKREMILKRKEEEALKDSLRPKLSEEQQRIIAILLDAHHKTYDPTYSDFCQFRPPVRVNDGGGSHPSRPNSRHTPSFSGDSSSSCSDHCITSSDMMDSSSFSNLDLSEEDSDDPSVTLELSQLSMLPHLADLVSYSIQKVIGFAKMIPGFRDLTSEDQIVLLKSSAIEVIMLRSNESFTMDDMSWTCGNQDYKYRVSDVTKAGHSLELIEPLIKFQVGLKKLNLHEEEHVLLMAICIVSPDRPGVQDAALIEAIQDRLSNTLQTYIRCRHPPPGSHLLYAKMIQKLADLRSLNEEHSKQYRCLSFQPECSMKLTPLVLEVFGNEIS

SEQ ID NO: 3 - DNA, 3393 bp, Homo sapiens
atggcggcagccgttctgagtgggccctctgcgggctccgcggctggggttcctggcgggaccgggggtctctcggcagtgagctcgggcccgcggctccgcctgctgctgctggagagtgtttctggtttgctgcaacctcgaacggggtctgccgttgctccggtgcatcccccaaaccgctcggccccacatttgcccgggctcatgtgcctattgcggctgcatgggtcggtgggcggggcccagaacctttcagctcttggggcattggtgagtctcagtaatgcacgtctcagttccatcaaaactcggtttgagggcctgtgtctgctgtccctgctggtaggggagagccccacagagctattccagcagcactgtgtgtcttggcttcggagcattcagcaggtgttacagacccaggacccgcctgccacaatggagctggccgtggctgtcctgagggacctcctccgatatgcagcccagctgcctgcactgttccgggacatctccatgaaccacctccctggccttctcacctccctgctgggcctcaggccagagtgtgagcagtcagcattggaaggaatgaaggcttgtatgacctatttccctcgggcttgtggttctctcaaaggcaagctggcctcattttttctgtctagggtggatgccttgagccctcagctccaacagttggcctgtgagtgttattcccggctgccctctttaggggctggcttttcccaaggcctgaagcacaccgagagctgggagcaggagctacacagtctgctggcctcactgcacaccctgctgggggccctgtacgagggagcagagactgctcctgtgcagaatgaaggccctggggtggagatgctgctgtcctcagaagatggtgatgcccatgtccttctccagcttcggcagaggttttcgggactggcccgctgcctagggctcatgctcagctctgagtttggagctcccgtgtccgtccctgtgcaggaaatcctggatttcatctgccggaccctcagcgtcagtagcaagaatattagcttgcatggagatggtcccctgcggctgctgctgctgccctctatccaccttgaggccttggacctgctgtctgcactcatcctcgcgtgtggaagccggctcttgcgctttgggatcctgatcggccgcctgcttccccaggtcctcaattcctggagcatcggtagagattccctctctccaggccaggagaggccttacagcacggttcggaccaaggtgtatgcgatattagagctgtgggtgcaggtttgtggggcctcggcgggaatgcttcagggaggagcctctggagaggccctgctcacccacctgctcagcgacatctccccgccagctgatgcccttaagctgcgtagcccgcgggggagccctgatgggagtttgcagactgggaagcctagcgcccccaagaagctaaagctggatgtgggggaagctatggccccgccaagccaccggaaaggggatagcaatgccaacagcgacgtgtgtgcggctgcactcagaggcctcagccggaccatcctcatgtgtgggcctctcatcaaggaggagactcacaggagactgcatgacctggtcctccccctggtcatgggtgtacagcagggtgaggtcctaggcagctccccgtacacgagctcccgctgccgccgtgaactctactgcctgctgctggcgctgctgctggccccgtctcctcgctgcccacctcctcttgcctgtgccctgcaagccttctccctcggccagcgagaagatagccttgaggtctcctctttctgctcagaagcactggtgacctgtgctgctctgacccacccccgggttcctcccctgcagcccatgggccccacctgccccacacctgctccagttccccctcctgaggccccatcgcccttcagggccccaccgttccatcctccgggccccatgccctcagtgggctccatgccctcagcaggccccatgccctcagcaggccccatgccctcagcaggccctgtgccctcggcacgccctggacctcccaccacagccaaccacctaggcctttctgtcccaggcctagtgtctgtccctccccggcttcttcctggccctgagaaccaccgggcaggctcaaatgaggaccccatccttgcccctagtgggactcccccacctactatacccccagatgaaacttttggggggagagtgcccagaccagcctttgtccactatgacaaggaggaggcatctgatgtggagatctccttggaaagtgactctgatgacagcgtggtgatcgtgcccgaggggcttccccccctgccacccccaccaccctcaggtgccacaccaccccctatagcccccactgggccaccaacagcctcccctcctgtgccagcgaaggaggagcctgaagaacttcctgcagccccagggcctctcccgccacccccacctccgccgccgcctgttcctggtcctgtgacgctccctccaccccagttggtccctgaagggactcctggtgggggaggacccccagccctggaagaggatttgacagttattaatatcaacagcagtgatgaagaggaggaggaagaggaagaaggggaagaagaagaagaggaagaagaggaagaggaggaagactttgaggaagaggaagaggatgaagaggaatattttgaagaggaagaagaggaggaagaagagtttgaggaagaatttgaggaagaagaaggtgagttagaggaagaagaagaagaggaggatgaggaggaggaagaagaactggaagaggtggaagacctggagtttggcacagcaggaggggaggtagaagaaggtgcacctccacccccaaccctgcctccagctctgcctccccctgagtctcccccaaaggtgcagccagaacccgaacccgaacccgggctgcttttggaagtggaggagccagggacggaggaggagcgtggggctgacacagctcccaccctggcccctgaagcgctcccctcccagggagaggtggagagggaaggggaaagccctgcggcagggccccctccccaggagcttgttgaagaagagccctctgctcccccaaccctgttggaagaggagcctgaggatgggagtgacaaggtgcagcccccaccagagacacctgcagaagaagagatggagacagagacagaggccgaagctctccaggaaaaggagcaggatgacacagctgccatgctggccgacttcatcgattgtccccctgatgatgagaagccaccacctcccacagagcctgactcctag

SEQ ID NO: 4 - protein, 1130 aa, Homo sapiens
MAAAVLSGPSAGSAAGVPGGTGGLSAVSSGPRLRLLLLESVSGLLQPRTGSAVAPVHPPNRSAPHLPGLMCLLRLHGSVGGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGASAGMLQGGASGEALLTHLLSDISPPADALKLRSPRGSPDGSLQTGKPSAPKKLKLDVGEAMAPPSHRKGDSNANSDVCAAALRGLSRTILMCGPLIKEETHRRLHDLVLPLVMGVQQGEVLGSSPYTSSRCRRELYCLLLALLLAPSPRCPPPLACALQAFSLGQREDSLEVSSFCSEALVTCAALTHPRVPPLQPMGPTCPTPAPVPPPEAPSPFRAPPFHPPGPMPSVGSMPSAGPMPSAGPMPSAGPVPSARPGPPTTANHLGLSVPGLVSVPPRLLPGPENHRAGSNEDPILAPSGTPPPTIPPDETFGGRVPRPAFVHYDKEEASDVEISLESDSDDSVVIVPEGLPPLPPPPPSGATPPPIAPTGPPTASPPVPAKEEPEELPAAPGPLPPPPPPPPPVPGPVTLPPPQLVPEGTPGGGGPPALEEDLTVININSSDEEEEEEEEGEEEEEEEEEEEEDFEEEEEDEEEYFEEEEEEEEEFEEEFEEEEGELEEEEEEEDEEEEEELEEVEDLEFGTAGGEVEEGAPPPPTLPPALPPPESPPKVQPEPEPEPGLLLEVEEPGTEEERGADTAPTLAPEALPSQGEVEREGESPAAGPPPQELVEEEPSAPPTLLEEEPEDGSDKVQPPPETPAEEEMETETEAEALQEKEQDDTAAMLADFIDCPPDDEKPPPPTEPDS

SEQ ID NO: 5 - DNA, 498 bp, Homo sapiens
cgcagccaccgagacaccatgagagccctcacactcctcgccctattggccctggccgcactttgcatcgctggccaggcaggtgcgaagcccagcggtgcagagtccagcaaaggtgcagcctttgtgtccaagcaggagggcagcgaggtagtgaagagacccaggcgctacctgtatcaatggctgggagccccagtcccctacccggatcccctggagcccaggagggaggtgtgtgagctcaatccggactgtgacgagttggctgaccacatcggctttcaggaggcctatcggcgcttctacggcccggtctagggtgtcgctctgctggcctggccggcaaccccagttctgctcctctccaggcacccttctttcctcttccccttgcccttgccctgacctcccagccctatggatgtggggtccccatcatcccagctgctcccaaataaactccagaagaggaatctgaaaaaaaaaaaaaaaaaa

SEQ ID NO: 6 - protein, 100 aa, Homo sapiens
MRALTLLALLALAALCIAGQAGAKPSGAESSKGAAFVSKQEGSEVVKRPRRYLYQWLGAPVPYPDPLEPRREVCELNPDCDELADHIGFQEAYRRFYGPV

SEQ ID NO: 7 - DNA, 2415 bp, Rattus norvegicus
cacggcgctccttagggccaccgctcggcgcgccgggacagaccctccccactcctgcctgcaggatcggaacgtcaattaacggctgacactgccccccacctcttcccacccatctgggctccagcgaggaacggatctcggggtacaccatgatcttgccatttttagtactggccatcggaccctgccttaccaactcatttgtgccagagaaagagaaagaccccagttactggcgacagcaagcccaagagaccttgaaaaatgccctgaaactccaaaaactcaacaccaacgtggccaagaacatcatcatgttcctgggagatggtatgggcgtctccacagtgacagctgcccgcatccttaagggccagctacaccacaacacgggcgaggagacacggctggagatggacaagttcccctttgtggctctctccaagacgtacaacaccaacgctcaggtccccgacagcgcgggcactgccactgcctacttgtgtggcgtgaaggccaacgagggcaccgtgggagtgagcgcggccactgagcgcacgcgatgcaacaccactcaggggaacgaggtcacgtccatcctgcgctgggccaaggatgctgggaagtccgtgggcatcgtgaccaccactcgggtgaaccacgccactcccagtgcagcctatgcgcactcggccgatcgggactggtactcggacaatgagatgcgcccagaggctctgagccagggctgcaaggacatcgcctatcagctaatgcacaacatcaaggacatcgatgtgatcatgggtggtggccggaagtacatgtaccccaagaacagaactgatgtggaatatgaactggatgagaaggccaggggcaccagactggatggcctggacctcatcagcatttggaagagcttcaaacctagacacaagcactcccactatgtctggaaccgcactgaactgctggcccttgacccctccagggtggactacctcttaggtctctttgagcccggggacatgcagtatgagttgaatcggaacaacctgactgacccttccctctcggagatggtggaggtggccctccggatcctgacaaagaatcccaaaggcttcttcttgctagtggaaggaggcaggattgaccacgggcaccatgaaggcaaggccaagcaggcgctgcatgaggccgtggagatggatgaggccatcggaaaggcgggcaccatgacttcccagaaagacacgttgactgtggttactgctgatcactcccacgttttcacgtttggtggctacacccccaggggcaactccatttttggtctggctcccatggtgagcgacacggacaagaagcccttcacagccatcctgtatggcaacgggcctggttacaaggtggtggacggtgaacgggagaacgtctccatggtggattatgctcacaacaactaccaggcccagtccgctgtccccctgcggcacgagacccacggtggggaagatgtggcggtctttgccaagggccctatggctcacctgcttcacggcgtccatgagcagaactacatcccccacgtcatggcgtatgcctcctgcattggagccaaccttgaccactgtgcctgggccagctctgcgagcagcccctccccaggggccctgctgcttccactggctctgttccccctacgcaccctgttctgagggcccaggtcccacaagagcccacaatggacagccggctcccctccctttgtggcctgccacctggccgcccacactcaacggggaggcccaggcaacctcgagcaggaacagaagtttgctacctgcctcacttccgcccggaaccctccgtgggtcggattcctggctctgccgttgtttctctattcactgccttttggccagcagggtgggtttctctcttgggccggcaggacacagactgcgcagattcccaaagcaccttatttttctaccaaatatactctccagaccctgcaaccatcatggaacattccagatctgaccttctctcccctaccccttctctctggaacactgggtcccatagtcacagccagtccctcaacccaaccctccttggagggaagaccaggtctgctcagggtgagactcccaggaagccacctccggggttggctgtctacccagggtggccaggctgggaagaacaacccagccggacaggacgcacacactccccacccagctccagagactcgccaacccttcactgaagcgactcccctgtttggaatagcaaaaaaaaaaagaaagaaaaaaaagaaaaaaattttaatttctctttttggtgttggttaaaagggaacacaagacatttaaataaaatgttccaaataaaaaa

SEQ ID NO: 8 - protein, 523 aa, Rattus norvegicus
MILPFLVLAIGPCLTNSFVPEKEKDPSYWRQQAQETLKNALKLQKLNTNVAKNIIMFLGDGMGVSTVTAARILKGQLHHNTGEETRLEMDKFPFVALSKTYNTNAQVPDSAGTATAYLCGVKANEGTVGVSAATERTRCNTTQGNEVTSILRWAKDAGKSVGIVTTTRVNHATPSAAYAHSADRDWYSDNEMRPEALSQGCKDIAYQLMHNIKDIDVIMGGGRKYMYPKNRTDVEYELDEKARGTRLDGLDLISIWKSFKPRHKHSHYVWNRTELLALDPSRVDYLLGLFEPGDMQYELNRNNLTDPSLSEMVEVALRILTKNPKGFFLLVEGGRIDHGHHEGKAKQALHEAVEMDEAIGKAGTMTSQKDTLTVVTADHSHVFTFGGYTPRGNSIFGLAPMVSDTDKKPFTAILYGNGPGYKVVDGERENVSMVDYAHNNYQAQSAVPLRHETHGGEDVAVFAKGPMAHLLHGVHEQNYIPHVMAYASCIGANLDHCAWASSASSPSPGALLLPLALFPLRTL